(19) United States
(12) Patent Application Publication
(10) Pub. No.: US 2022/0275335 A1
(43) Pub. Date: Sep. 1, 2022

(54) CULTURE MEDIUM FOR HAEMATOPOIETIC INDUCTION

(71) Applicant: Adaptimmune Limited, Abingdon Oxfordshire (GB)

(72) Inventors: Cheng Tao Yang, Abingdon Oxfordshire (GB); Lee Carpenter, Abingdon Oxfordshire (GB)

(21) Appl. No.: 17/630,880

(22) PCT Filed: Aug. 20, 2020
(86) PCT No.: PCT/EP2020/073407

§ 371 Date: Jan. 27, 2022

(30) Foreign Application Priority Data
Aug. 20, 2019 (GB) 1911955.1
Aug. 7, 2020 (GB) 2012306.3

(51) Int. Cl. C12N 5/0789; A61K 35/17

(52) U.S. Cl. CPC C12N 5/0647; A61K 35/17; C12N 2501/105; C12N 2501/125; C12N 2501/145; C12N 2501/15; C12N 2501/155; C12N 2501/2303; C12N 2501/2306; C12N 2501/2307; C12N 2501/26

(57) ABSTRACT

This invention concerns chemically defined haematopoietic induction media that support the differentiation of haemogenic endothelial cells (HECs) into haematopoietic progenitor cells (MFCs) that are capable of further differentiation into T cells. The media may (a) stimulate cKIT receptor and/or cKIT receptor mediated signalling pathways and/or (b) stimulate VEGFR and/or VEGFR mediated signalling pathways. For example, the medium may comprise SCF and VEGF. In some embodiments, the media may further (c) stimulate MPL or MPL mediated signalling pathways; (d) stimulate FLT3 or FLT3 mediated signalling pathways (e) stimulate IGF1R or IGF1R mediated signalling pathways and (f) display interleukin (IL) activity. For example, the medium may further comprise Thrombopoietin (TPO), Fits ligand (FltSL), IGF-1, IL-3, IL-6 and optionally IL-7. These media may be useful for example in the production of blood cells or use in immunotherapy.

### FIELD

This invention relates to culture media for supporting the differentiation of mammalian induced pluripotent stem cells (iPSCs) into Haematopoietic Progenitor Cells (HPCs).

### BACKGROUND

Immunotherapeutics are poised to transform the cancer treatment landscape with the promise of long-term survival (McDermott et al., Cancer Treat Rev. 2014 October; 40(9): 1056-64). There is a clear unmet medical need for new immunomodulatory drugs to expand patient population and range of tumor types. In addition, new agents are needed to enhance the magnitude and duration of anti-tumor responses. The development of these agents has been possible because of the in-depth understanding of the basic principles controlling T-cell immunity over the last two decades (Sharma and Allison, Cell. 2015 April 9; 161(2): 205-14). This typically requires tumor specific CD4+ and CD8+ T-cells recognising tumor-associated peptide antigens presented by MHC molecules. Different vaccination strategies and adoptive transfer of ex vivo expanded tumor infiltrated lymphocytes have in some cases demonstrated the ability of tumor specific T-cells to treat late stage cancer (Rosenberg et al., Nat Med. 2004 September; 10(9): 909-15).

However, current adoptive T cell therapies are limited by a lack of suitable patient and tumor-specific T cells and there is a need for therapeutically sufficient and functional antigen-specific T cells for effective use in immunotherapy.

The differentiation of induced pluripotent stem cells (iPSCs) into HPCs and T cells has been reported (Kennedy et al 2012 Cell Reports 2 6 1722-1735; Sturgeon et al 2014 Nat Biotechnol 32(6) 554-561; Ditadi et al (2015) Nat Cell Biol.;17(5):580-91). Typically this involves the use of embryoid body formation and/or serum, and, in later stages, the use of OP9-DLL4 stroma, for the end stage of T cell differentiation (also see Themeli et al (2013) Nat Biotechnol. 31(10):928-33; Vizcardo et al 2013 Cell Stem Cell. 2013 Jan. 3;12(1):31-6.). Cells produced using media that contain feeder, stromal cells, serum or other undefined components are not suitable for clinical applications.

### SUMMARY

The present inventors have developed chemically defined haematopoietic induction media that support the differentiation of haemogenic endothelial cells (HECs) into HPCs capable of further differentiation into T cells. These media (referred to as SV, HEM7.2 and HEM7.3) may allow the isolation of HPCs at much later time points (d16-18), in a serum free and directed GMP compliant manner that permits onward culture towards the T cell lineage without the use of OP9-D14 stroma, at a reduced costs relative to existing haematopoietic induction media. This may be useful for example in the production of clinical grade blood cells, such as T cells, for use in immunotherapy.

A first aspect of the invention provides a method of producing a population of HPCs comprising (i) culturing a population of HECs in a haematopoietic induction medium to produce a population of HPCs,
wherein the haematopoietic induction medium is a chemically defined medium that (a) stimulates cKIT receptor (CD117; KIT receptor tyrosine kinase) and/or cKIT receptor (CD117; KIT receptor tyrosine kinase) mediated signalling pathways and/or (b) stimulates VEGFR and/or VEGFR mediated signalling pathways.

Preferably, the haematopoietic induction medium both (a) stimulates cKIT receptor (CD117; KIT receptor tyrosine kinase) and/or cKIT receptor mediated signalling pathways and (b) stimulates VEGFR and/or VEGFR mediated signalling pathways.

The haematopoietic induction medium may comprise a differentiation factor that stimulates c-KIT receptor (CD117) or c-KIT receptor (CD117) mediated signalling pathways, such as SCF, and/or a differentiation factor that stimulates VEGFR or VEGFR mediated signalling pathways, preferably VEGFR2 or VEGFR2 mediated signalling pathways, such as VEGF-A.

Preferably, these are the only differentiation factors in the medium.

A preferred haematopoietic induction medium of the first aspect may comprise SCF and/or VEGF. For example, the haematopoietic induction medium may consist of a chemically defined nutrient medium supplemented with one or more differentiation factors, wherein the one or more differentiation factors consist of SCF and/or VEGF.

A second aspect of the invention provides a method of producing a population of HPCs comprising
(i) culturing a population of HECs in a haematopoietic induction medium to produce a population of HPCs,
wherein the haematopoietic induction medium is a chemically defined medium that (a) stimulates cKIT receptor (CD117) and/or cKIT receptor (CD117) mediated signalling pathways, (b) stimulates VEGFR and/or VEGFR mediated signalling pathways, (c) stimulates MPL (CD110) and/or MPL (CD110) mediated signalling pathways (d) stimulates FLT3 and/or FLT3 mediated signalling pathways (e) stimulates IGF1R and/or IGF1R mediated signalling pathways and (0 displays interleukin (IL) activity.

A preferred haematopoietic induction medium of the second aspect may comprise VEGF, SCF, Thrombopoietin (TPO), FIt3 ligand (FIt3L), IL-3, IL-6, IGF-1 and optionally IL-7. For example, the haematopoietic induction medium may consist of a chemically defined nutrient medium supplemented with one or more differentiation factors, wherein the one or more differentiation factors consist of VEGF, SCF, Thrombopoietin (TPO), Flt3 ligand (FIt3L), IL-3, IL-6, IGF-1 and optionally IL-7.

The HPCs produced by the methods of the first and second aspects may be further differentiated to produce T cells. For example, a method of the first aspect may further comprise;
(ii) differentiating the population of HPCs into T cell progenitor cells; and
(iii) maturing the progenitor T cells to produce a population of double positive CD4+CD8+T cells.

A method of the first or second aspect may further comprise
(iv) activating and expanding the double positive CD4+CD8+ T cells to produce a population of single positive CD8+ T cells or a population of single positive CD4+ T cells.

In preferred embodiments, HECs for use in the methods of the first aspect may be produced by differentiating a population of iPSCs into HECs.

A third aspect of the invention provides a haematopoietic induction medium comprising a chemically defined nutrient medium supplemented with one or more differentiation factors, wherein the one or more differentiation factors consist of a differentiation factor that stimulates cKIT receptor (CD117) or cKIT receptor (CD117) mediated signalling pathways, such as SCF, and/or a differentiation factor that stimulates VEGFR (optionally VEGFR2) or VEGFR (optionally VEGFR2) mediated signalling pathways, such as VEGF (optionally VEGF-A).

A fourth aspect of the invention provides a haematopoietic induction medium comprising a chemically defined nutrient medium supplemented with one or more differentiation factors, wherein the one or more differentiation factors consist of a differentiation factor that stimulates cKIT receptor (CD117) and/or cKIT receptor (CD117) mediated signalling pathways, such as SCF; a differentiation factor that stimulates VEGFR and/or VEGFR mediated signalling pathways, such as VEGF; a differentiation factor that stimulates MPL (CD110) and/or MPL (CD110) mediated signalling pathways, such as TPO; a differentiation factor that stimulates FLT3 and/or FLT3 mediated signalling pathways, such as Flt3L; a differentiation factor that stimulates IGF1R and/or IGF1R mediated signalling pathways, such as IGF-1, and; one or more differentiation factors that display interleukin (IL) activity, for example, one or more interleukins, such as IL-3, IL-6 and optionally IL-7.

A fifth aspect of the invention provides the use of a haematopoietic induction medium of the third or fourth aspect in the differentiation of HECs into HPCs.

A sixth aspect of the invention provides a kit for the production of HPCs comprising a haematopoietic induction medium of the third or fourth aspect.

The kit may further comprise suitable media to support the differentiation of iPSCs into HECs. For example, the kit may further comprise;
a first mesoderm induction medium comprising activin,
a second mesoderm induction medium comprising activin, BMP and FGF, and
a third mesoderm induction medium comprising activin, BMP, FGF, and a GSK3 inhibitor.

These and other aspects and embodiments of the invention are described in more detail below.

### BRIEF DESCRIPTION OF FIGURES

FIG. 1 shows a schematic view of an example of a six-stage method for generating T cells from iPSCs.

FIG. 2 shows the characterisation of CD34+ CD45+ CD7+ cell emergence from differentiating hiPSCs: The emergence of CD34+ CD45+ CD7+ progenitors is shown across 3 separate lines, ChiPSC31 (2A), ADAP-J (2B) and NIH2 (2C), with both full (HEM7) and minimal (SV) Stage 3 conditions. Typically, cells are isolated from the monolayers at different time points, stained with fluorescent antibodies and assessed by flow cytometry. CD34+ CD45+ CD7+ progenitors are shown to emerge under full (HEM7) and minimal (SV) Stage 3 conditions from d16-d28.

FIG. 3 shows the number of CD45+ CD7+ CD5+ T cell progenitors produced from CD34+ or CD34+ CD7+ HPCs produced from ChiPSC31 (n=3; 3A) and NIH2 (n=2; 3B) cell lines using a haematopoietic induction medium described herein (“minimal Stage 3 media”). Cells were assessed after staining with appropriate antibodies and flow cytometry. Cells numbers indicate number of proT cells generated per well/24WP, with 5×10³ cells seeded on d0 of the LP culture stage (Stage 4).

FIG. 4 shows the number of CD3+, CD4+ and CD8+ double positive T cells produced from CD34+ or CD34+ CD7+ HPCs produced from ChiPSC31 (n=3; 3A) and NIH2 (n=2; 3B) cell lines using a haematopoietic induction medium described herein (“minimal Stage 3 media”). Cells were assessed after staining with appropriate antibodies and flow cytometry. Cells numbers indicate number to single positive T cells generated per well/24WP, with 5×10³ cells seeded on d0 of the LP culture stage.

FIG. 5 shows antigen-specific killing measured using a KILR™ assay by T cells generated from differentiation using full (HEM7) and minimal (SV) Stage 3 conditions.

FIG. 6 shows the emergence of CD34+CD45+CD7+ progenitors from hiPSC line NIH2, with both standard (HEM7) and modified (HEM7.2) Stage 3 conditions. Typically cells are isolated from the monolayers on day 16, stained with fluorescent antibodies and assessed by flow cytometry. CD34⁺CD45⁺CD7⁺ progenitors are shown to emerge under HEM7 (FIGS. 6A and 6B left panel) and HEM7.2 Stage 3 conditions (FIGS. 6A and 6B right panel). Output from NIH2 cell line (n3) was shown

FIG. 7 shows that isolation of CD34⁺ HPCs and onward culture demonstrates proT cell potential. CD34⁺ HPC were isolated from hiPSCs differentiated with standard (HEM7; FIG. 7A left panel) and new (HEM7.2; FIG. 7A right panel) Stage 3 media, and further cultured in SCT's lymphoid proliferation media (LP) media, that they are capable of generating CD7⁺ and CD5⁺ proT cells, as assessed after staining with appropriate antibodies and flow cytometry. Output from NIH2 cell line (n2) was shown

FIG. 8 shows that isolation of CD34⁺ HPC and onward culture demonstrates full T cell potential. CD34⁺ HPCs were isolated from hiPSCs differentiated with standard (HEM7) and new (HEM7.2) Stage 3 media, and further cultured in SCT's lymphoid proliferation media (LP) media and T cell maturation (TM) media, that they were capable of generating CD4⁺ and CD8⁺ double positive T cells and expressed CD3, as assessed after staining with appropriate antibodies and flow cytometry. Output from NIH2 cell line (n2) was shown.

FIG. 9 shows antigen-specific killing by T cells generated from differentiation using HEM7 (FIG. 9A) or HEM7.2 (FIG. 9B). Isolated CD34⁺ HPCs from both standard (HEM7) and new (HEM7.2) Stage 3 conditions were harvested on day 16. Typically cells were cultured in SCT proprietary media for T cell proliferation (LP) media and then transduced with MAGE-A4 (TD) or Non transduction (NTD). The derived T cell were then cultured in T cell maturation (TM) media and further stage 6 media with activators (S6). Mature T cells were evaluated in KILR assay. The results show antigen-specific killing by T cells generated from standard (HEM7) and new (HEM7.2) Stage 3 conditions

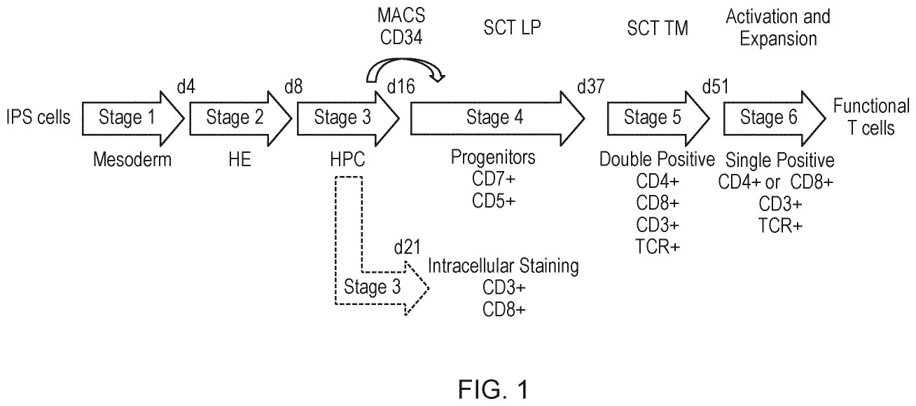

### DETAILED DESCRIPTION

This invention relates to the development of defined culture media comprising a defined set of differentiation factors that are capable of driving the differentiation of haemogenic endothelial cells (HECs) into haematopoietic progenitor cells (HPCs). For example, a medium of the invention (SV) may contain only two differentiation factors (SCF and VEGF); a medium of the invention (HEM7.2) may contain only seven differentiation factors (VEGF, SCF, Thrombopoietin (TPO), Flt3 ligand (FIt3L), IL-3, IL-6 and IGF-1); or a medium of the invention (HEM7.3) may contain only eight differentiation factors (VEGF, SCF, Thrombopoietin (TPO), Flt3 ligand (FIt3L), IL-3, IL-6, IGF-1 and IL-7). Existing haematopoietic induction media contain a large number of differentiation factors, making their use uneconomical and difficult to scale up. The use of fewer differentiation factors may make the media described herein more economical and easier to scale up. HPCs produced using the media of the invention may be useful, for example, in the production of T cells for use in immunotherapy.

HECs are partially differentiated endothelial progenitor cells that have hematopoietic potential and are capable of differentiation under appropriate conditions into haematopoietic lineages. HECs may express CD34. In some embodiments, HECs may not express CD73 or CXCR4 (CD184). For example, the HECs may have the phenotype CD34+CD73− or the phenotype CD34+CD73− CXCR4−. Suitable HECs may be isolated by CD34 selection.

HECs may be differentiated into HPCs by culturing the population of HECs in a haematopoietic induction medium of the invention.

A suitable haematopoietic induction medium may stimulate (i) cKIT receptor (CD117) or cKIT receptor (CD117) mediated signalling pathways and/or (ii) VEGFR or VEGFR mediated signalling pathways, preferably VEGFR2 or VEGFR2 mediated signalling pathways. For example, a haematopoietic induction medium may comprise the differentiation factors: VEGF-A and/or SCF.

Preferably the haematopoietic induction medium stimulates both (i) cKIT receptor (CD117) or cKIT receptor (CD117) mediated signalling pathways and (ii) VEGFR of VEGFR mediated signalling pathways, preferably VEGFR2 or VEGFR2 mediated signalling pathways. For example, the haematopoietic induction medium may comprise the differentiation factors: VEGF-A and SCF.

In some embodiments, a suitable haematopoietic induction medium may (a) stimulate cKIT receptor (CD117) and/or cKIT receptor (CD117) mediated signalling pathways, (b) stimulate VEGFR and/or VEGFR mediated signalling pathways, (c) stimulate MPL (CD110) and/or MPL (CD110) mediated signalling pathways (d) stimulate FLT3 and/or FLT3 mediated signalling pathways (e) stimulate IGF1R and/or IGF1R mediated signalling pathways and (f) display interleukin (IL) activity. For example, a haematopoietic induction medium may comprise the differentiation factors: VEGF, SCF, Thrombopoietin (TPO), Flt3 ligand (FIt3L), IGF-1, IL-3, and IL-6 (see Table 3); or a haematopoietic induction medium may comprise the differentiation factors: VEGF, SCF, Thrombopoietin (TPO), Flt3 ligand (FIt3L), IGF-1, IL-3, IL-6, and IL-7 (see Table 4).

Examples of suitable haematopoietic induction medium are shown in Table 3 (HEM7.2) and Table 4 (HEM7.3).

Vascular endothelial growth factor (VEGF) is a protein factor of the PDGF family which binds to VEGFR tyrosine kinase receptors and stimulates vasculogenesis and angiogenesis. Suitable VEGFs include any member of the VEGF family, for example any one of VEGF-A to VEGF-D and PGF. Preferably, the VEGF is VEGF-A (also known as VEGF, NCBI Gene ID: 7422, nucleic acid sequence NM_001025366.2, amino acid sequence NP_001020537.2). Preferably, the VEGFR mediated signalling pathways are VEGFR2 (KDR/Flk-1) mediated signalling pathways. VEGF is readily available from commercial sources (e.g. R&D Systems, USA). Conveniently, the concentration of VEGF in an HE induction medium described herein may be from 1 to 100 ng/ml, for example any of about 5, 7, 10, 12, 15, 17, 20, 25, 30, 35, 40, 45 or 50 ng/ml, preferably about 15 ng/ml.

In some examples of haematopoietic induction media, VEGF may be replaced by a VEGF activator or agonist that stimulates VEGFR or VEGFR mediated signalling pathways. Suitable VEGF activators are known in the art and include proteins, such as gremlin (Mitola et al (2010) Blood 116(18) 3677-3680) nucleic acids, such as shRNA (e.g. Turunen et al Circ Res. 2009 Sep. 11; 105(6):604-9), CRISPR-based plasmids (e.g. VEGF CRISPR activation plasmid; Santa Cruz Biotech, USA), antibodies and small molecules.

Stem cell factor (SCF) is a cytokine that binds to the cKIT receptor (KIT receptor tyrosine kinase; CD117; SCFR) and is involved in haematopoiesis. SCF (also called KITLG, NCBI GenelD: 4254) may have the reference nucleic acid sequence NM_000899.5 or NM_03994.5 and the reference amino acid sequence NP_000890.1 or NP_003985.5. SCF is readily available from commercial sources (e.g. R&D Systems, USA). Conveniently, the concentration of SCF in an HE induction medium described herein may be from 1 to 1000 ng/ml, for example any of about 10, 20, 30, 40, 50, 60, 70, 80, 90, 100, 110, 120, 130, 140, 150, 200, 250, 300, 350, 400, 450, 500, 600, 700, 800, 900 ng/ml, preferably about 100 ng/ml.

Thrombopoietin (TPO) is a glycoprotein hormone that regulates platelet production. TPO (also called THPO, NCBI Gene ID: 7066) may have the reference nucleic acid sequence NM_000460.4 and the reference amino acid sequence NP_000451.1. TPO is readily available from commercial sources (e.g. R&D Systems, USA; Miltenyi Biotec Gmbh, DE). Conveniently, the concentration of TPO in a haematopoietic induction medium described herein may be from 3 to 300 ng/ml, for example any of about 3, 4, 5, 6, 7, 8, 9, 10, 11, 12, 13, 14, 15, 20, 25, 27, 30, 32, 35, 40, 45, 50, 60, 70, 80, 90 or 100, 110, 120, 130, 140, 150, 160, 170, 180, 190, 200, 225, 250, 275 or 300 ng/ml, preferably about 30 ng/ml. Flt3 ligand (Fms-related tyrosine kinase 3 ligand or FLT3L) is a cytokine with haematopoietic activity which binds to the FLT3 receptor and stimulates the proliferation and differentiation of progenitor cells. Flt3 ligand (also called FLT3LG, NCBI GenelD: 2323) may have the reference nucleic acid sequence NM_001204502.2 and the reference amino acid sequence NP_001191431.1. Flt3 is readily available from commercial sources (e.g. R&D Systems, USA; Miltenyi Biotec Gmbh, DE). Conveniently, the concentration of Flt3 ligand in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

Interleukins (ILs) are cytokines that play major roles in immune development and function. ILs in a haematopoietic induction medium may include IL-3, IL-6, and IL-7.

IL-3 (also called IL3 or MCGF, NCBI GenelD: 3562) may have the reference nucleic acid sequence NM_000588.4 and the reference amino acid sequence NP_000579.2. IL-3 is readily available from commercial sources (e.g. R&D Systems, USA; Miltenyi Biotec Gmbh, DE). Conveniently, the concentration of IL-3 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

IL-6 (also called IL6 or HGF, NCBI GenelD: 3569) may have the reference nucleic acid sequence NM_000600.5 and the reference amino acid sequence NP_000591.5. IL-6 is readily available from commercial sources (e.g. R&D Systems, USA; Miltenyi Biotec Gmbh, DE). Conveniently, the concentration of IL-6 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-7 (also called IL7, NCBI GenelD: 3574) may have the reference nucleic acid sequence NM_000880.4 and the reference amino acid sequence NP_000871.1. IL-7 is readily available from commercial sources (e.g. R&D Systems, USA; Miltenyi Biotec Gmbh, DE). Conveniently, the concentration of IL-7 in a haematopoietic induction medium described herein may be from 0.1 to 100 ng/ml, for example any of about 0.1, 0.25, 0.5, 0.75, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

Insulin-like growth factor 1 (IGF-1) is a hormone that binds to the tyrosine kinases IGF-1 receptor (IGF1R) and insulin receptor and activates the multiple signalling pathways. IGF-1(also called IGF or MGF, NCBI GenelD: 3479) may have the reference nucleic acid sequence NM_000618.5 and the reference amino acid sequence NP_000609.1. IGF-1 is readily available from commercial sources (e.g. R&D Systems, USA). Conveniently, the concentration of IGF-1 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

A suitable haematopoietic induction medium may lack differentiation factors that stimulate (a) SMAD1, 5 and 9 mediated signalling pathways, for example a Bone Morphogenic Protein (BMP), such as BMP4; (b) Hedgehog signalling pathways, such as Sonic hedgehog (SHH) (c) the EpoR mediated signalling pathway, such as erythropoietin (EPO); and/or (d) AGTR2 mediated signalling pathways, such as angiotensin. A suitable haematopoietic induction medium may also lack differentiation factors that inhibit the AGTR1 (angiotensin II type 1 receptor (AT₁)) mediated signaling pathway, for example Angiotensin II type 1 receptor (AT₁) antagonists (ARBs), such as losartan (2-Butyl-4-chloro-1-{[2′-(1H-tetrazol-5-yl)-4-biphenylyl]methyl}-1H-imidazol-5-yl)methanol); and differentiation factors with fibroblast growth factor (FGF) activity, for example FGF, such as bFGF (FGF2). For example, a haematopoietic induction medium may be devoid of BMP, FGF. SHH, EPO; angiotensin and losartan.

Preferably, the haematopoietic induction medium is a chemically defined medium. For example, the haematopoietic induction medium may consist of a chemically defined nutrient medium supplemented with effective amounts of VEGF, for example 15 ng/ml; and SCF, for example 100 ng/ml. Other examples of the haematopoietic induction medium may consist of a chemically defined nutrient medium supplemented with effective amounts of VEGF, for example 15 ng/ml; SCF, for example 100 ng/ml; thrombopoietin (TPO), for example 30 ng/ml; Flt3 ligand (FIt3L), for example 25 ng/ml; IL-3, for example 25 ng/ml; IL-6, for example 10 ng/ml; IGF-1, for example 25 ng/ml; and optionally IL-7, for example 10 ng/ml. A suitable haematopoietic induction medium be devoid of other differentiation factors. For example, a haematopoietic induction medium may consist of a chemically defined nutrient medium supplemented with one or more differentiation factors, wherein the one or more differentiation factors consist of SCF and/or VEGF (i.e. the medium does not contain any differentiation factors other than SCF and VEGF). Other examples of a haematopoietic induction medium may consist of a chemically defined nutrient medium supplemented with one or more differentiation factors, wherein the one or more differentiation factors consist of VEGF, SCF, Thrombopoietin (TPO), Flt3 ligand (FIt3L), IL-3, IL-6, IGF-1 and IL-7 (i.e. the medium does not contain any differentiation factors other than VEGF, SCF, Thrombopoietin (TPO), Flt3 ligand (FIt3L), IL-3, IL-6 and IGF-1; or VEGF, SCF, Thrombopoietin (TPO), Flt3 ligand (FIt3L), IL-3, IL-6, IGF-1 and IL-7).

Suitable chemically defined nutrient media are described below and include StemPro™-34 PLUS (ThermoFisher Scientific) or a basal medium such as IMDM supplemented with albumin, insulin, selenium transferrin, and lipids as described below.

The HECs may be cultured in the haematopoietic induction medium for 8-35 days, preferably 12-28 days or 16-28 days, for example any of 13, 14, 15, 16, 17, 18, 19, 20, 21, 22, 23, 24, 25, 26 or 27 days, for example about 16 days, to produce the population of HPCs.

HPCs (also called hematopoietic stem and progenitor cells or HSPCs) are multipotent stem cells that are committed to a hematopoietic lineage and are capable of further hematopoietic differentiation into all blood cell types including myeloid and lymphoid lineages, including monocytes, B cells, NK cells, NKT cells and T cells. HPCs may express CD34 i.e. the HPCs may display a CD34+ phenotype.

In some embodiments, HPCs may include thymocytic HPCs (tHPCs). Thymocytic HPCs are progenitor cells that are committed to the thymocytic lineage (i.e. early stage thymocytic cells) and capable of further differentiation into T cells. tHPCs may express CD34 and CD7 i.e. the tHPCs may display a CD34+CD7+ phenotype.

HPCs may co-express CD117, CD133, CD45 and FLK1 (also known as KDR or VEGFR2) and may be negative for expression of CD38 and other lineage specific markers. For example, HPCs may display one or more, preferably all of CD34+ CD133+ CD45+ FLK1+ CD38−. Thymocytic HPCs may display one or more, preferably all of CD34+ CD7+ CD133+ CD45+ FLK1+ CD38−.

A method described herein may further comprise providing a population of HECs for use in a method of producing HPCs as described herein. In some preferred embodiments, HECs for use in the methods described herein are produced in vitro from induced pluripotent stem cells (iPSCs). For example, iPSCs may be differentiated into HECs using a two-step process that includes a mesoderm stage. For example, a method may comprise;
(i) differentiating the population of iPSCs into mesoderm cells, and
(ii) differentiating the mesoderm cells into HECs.

The method may further comprise (iii) differentiating the HECs into a population of HPCs using a haematopoietic induction medium as described herein.

Induced pluripotent stem cells (iPSCs) are pluripotent cells which are derived from non-pluripotent, fully differentiated donor or antecedent cells. iPSCs are capable of self-renewal in vitro and exhibit an undifferentiated phenotype and are potentially capable of differentiating into any foetal or adult cell type of any of the three germ layers (endoderm, mesoderm and endoderm). The population of iPSCs may be clonal i.e. genetically identical cells descended from a single common ancestor cell.

iPSCs may express one or more of the following pluripotency associated markers: POU5f1 (Oct4), Sox2, Alkaline Phosphatase, SSEA-3, Nanog, SSEA-4, Tra-1-60, KLF4 and c-myc, preferably one or more of POU5f1, NANOG and SOX2. An iPSC may lack markers associated with specific differentiative fates, such as Brachury (T), Sox17, FoxA2, αFP, Sox1, NCAM, GATA6, GATA4, Hand1 and CDX2. In particular, an iPSC may lack markers associated with endodermal fates.

Preferably, the iPSCs are human IPSCs (hiPSCs).

In some embodiments, iPSCs may be gene edited, for example to inactivate or delete HLA genes or other genes associated with immunogenicity or GVHD, or optionally to include nucleic acids encoding exogenous antigen receptor, for example exogenous TCR, CAR or NKCR.

IPSCs may be derived or reprogramed from donor cells, which may be somatic cells or other antecedent cells obtained from a source, such as a donor individual. The donor cells may be mammalian, preferably human cells. Suitable donor cells include adult fibroblasts and blood cells, for example peripheral blood cells, such as HPCs or mononuclear cells.

Suitable donor cells for reprogramming into iPSCs as described herein may be obtained from a donor individual. In some embodiments, the donor individual may be the same person as the recipient individual to whom the T cells will be administered following production as described herein (autologous treatment). In other embodiments, the donor individual may be a different person to the recipient individual to whom the T cells will be administered following production as described herein (allogeneic treatment). For example, the donor individual may be a healthy individual who is human leukocyte antigen (HLA) matched (either before or after donation) with a recipient individual suffering from cancer. Alternatively, the donor individual may not be HLA matched with the recipient individual. Preferably, the donor individual may be a neonate (new-born), for example the donor cells may be obtained from a sample of umbilical cord blood.

Suitable donor individuals are preferably free of communicable viral (e.g. HIV, HPV, CMV) and adventitious agents (e.g. bacteria, mycoplasma), and free of known genetic abnormalities.

In some embodiments, a population of peripheral blood cells, such as HPCs, for reprogramming may be isolated from a blood sample, preferably an umbilical cord sample, obtained from the donor individual. Suitable methods for the isolation of HPCs and other peripheral blood cells, are well-known in the art and include, for example magnetic activated cell sorting (see, for example, Gaudernack et al 1986 J Immunol Methods 90 179), fluorescent activated cell sorting (FACS: see for example, Rheinherz et al (1979) PNAS 76 4061), and cell panning (see for example, Lum et al (1982) Cell Immunol 72 122). HPCs may be identified in a sample of blood cells by expression of CD34. In other embodiments, a population of fibroblasts for reprogramming may be isolated from a skin biopsy following dispersal using collagenase or trypsin and out-growth in appropriate cell culture conditions.

In some embodiments, iPSCs may be derived from antigen-specific T cells. For example, the T cells may comprise nucleic acid encoding TCRs, such as αβ TCRs, that bind to an antigen, such as a tumor antigen, displayed in complex with a class 1 MHC. Antigen-specific T cells for use in the generation of iPSCs may be obtained by screening a diverse population of T cells with peptide epitopes from the target antigen displayed on a class I or II MHC molecule on the surface of an antigen presenting cell, such as a dendritic cell, or by isolating from a tumour sample from a cancer patient.

Donor cells are typically reprogrammed into iPSCs by the introduction of reprogramming factors, such as Oct4, Sox2 and Klf4 into the cell. The reprogramming factors may be proteins or encoding nucleic acids and may be introduced into the differentiated cells by any suitable technique, including plasmid, transposon or more preferably, viral transfection or direct protein delivery. Other reprogramming factors, for example Klf genes, such as Klf-1, -2, -4 and -5; Myc genes such as C-myc, L-myc and N-myc; Nanog; SV40 Large T antigen; Lin28; and short hairpins (shRNA) targeting genes such as p53, may also be introduced into the cell to increase induction efficiency. Following introduction of the reprogramming factors, the donor cells may be cultured. Cells expressing pluripotency markers may be isolated and/or purified to produce a population of iPSCs. Techniques for the production of iPSCs are well-known in the art (Yamanaka et al Nature 2007; 448:313-7; Yamanaka 6 2007 Jun 7; 1(1):39-49; Kim et al Nature. 2008 Jul. 31; 454(7204):646-50; Takahashi Cell. 2007 Nov. 30; 131(5):861-72. Park et al Nature. 2008 Jan. 10; 451(7175):141-6; Kimet et al Cell Stem Cell. 2009 Jun. 5; 4(6):472-6; Vallier, L., et al. Stem Cells, 2009. 9999(999A): p. N/A; Baghbaderani et al 2016; Stem Cell Rev. 2016 August; 12(4):394-420; Baghbaderani et al. (2015) Stem Cell Reports, 5(4), 647-659).

Conventional techniques may be employed for the culture and maintenance of iPSCs (Vallier, L. et al Dev. Biol. 275, 403-421 (2004), Cowan, C.A. et al. N. Engl. J. Med. 350, 1353-1356 (2004), Joannides, A. et al. Stem Cells 24, 230-235 (2006) Klimanskaya, I. et al. Lancet 365, 1636-1641 (2005), Ludwig, T. E. et al. Nat. Biotechnol. 24, 185-187 (2006)). iPSCs for use in the present methods may be grown in defined conditions or on feeder cells. For example, iPSCs may be conventionally cultured in a culture dish on a layer of feeder cells, such as irradiated mouse embryonic fibroblasts (MEF), at an appropriate density (e.g. 10⁵ to 10⁶ cells/60 mm dish), or on an appropriate substrate, in a feeder conditioned or defined iPSC maintenance medium. iPSCs for use in the present methods may be passaged by enzymatic or mechanical means. In some embodiments, iPSCs may be passaged on matrigel™ or an ECM protein, such as vitronectin, in an iPSC maintenance medium, such as mTeSR™1 or TeSR™2 (StemCell Technologies) or E8 flex (Life Thermo) culture medium.

Differentiation and maturation of the cell populations in the steps of the methods described herein is induced by culturing the cells in a culture medium supplemented with a set of differentiation factors. The set of differentiation factors that is listed for each culture medium is preferably exhaustive and medium may be devoid of other differentiation factors. In preferred embodiments, the culture media are chemically defined media. For example, a culture medium may consist of a chemically defined nutrient medium that is supplemented with an effective amount of one or more differentiation factors, as described below. A chemically defined nutrient medium may comprise a basal medium that is supplemented with one or more serum-free culture medium supplements.

Differentiation factors are factors which modulate, for example promote or inhibit, a signalling pathway which mediates differentiation in a mammalian cell. Differentiation factors may include growth factors, cytokines and small molecules which modulate one or more of the Activin/Nodal, FGF, Wnt or BMP or signalling pathways thereof. Examples of differentiation factors include Activin/Nodal, FGFs, BMPs, retinoic acid, vascular endothelial growth factor (VEGF), stem cell factor (SCF), TGF6 ligands, GDFs, LIF, Interleukins, GSK-3 inhibitors, phosphatidylinositol 3-kinase (PI3K) inhibitors, Thrombopoietin (TPO), Flt3 ligand (FIt3L), IGF-1, and interleukins, such as IL-3, IL-6, and IL-7.

Differentiation factors which are used in one or more of the media described herein include TGFβ ligands, such as activin, fibroblast growth factor (FGF), bone morphogenetic protein (BMP), stem cell factor (SCF), vascular endothelial growth factor (VEGF), GSK-3 inhibitors (such as CHIR-99021), interleukins, and hormones, such as IGF-1, TPO and angiotensin II. A differentiation factor may be present in a medium described herein in an amount that is effective to modulate a signalling pathway in cells cultured in the medium.

In some embodiments, a differentiation factor listed above or below may be replaced in a culture medium by a factor that has the same effect (i.e. stimulation or inhibition) on the same signalling pathway. Suitable factors are known in the art and include proteins, nucleic acids, antibodies and small molecules.

The extent of differentiation of the cell population during each step may be determined by monitoring and/or detecting the expression of one or more cell markers in the population of differentiating cells. For example, an increase in the expression of markers characteristic of the more differentiated cell type or a decrease in the expression of markers characteristic of the less differentiated cell type may be determined. The expression of cell markers may be determined by any suitable technique, including immunocytochemistry, immunofluorescence, RT-PCR, immunoblotting, fluorescence activated cell sorting (FACS), and enzymatic analysis. For example, a cell which is stated herein not to express a marker may display active transcription and intracellular expression of the marker gene but detectable levels of the marker may not be present on the surface of the cell.

A population of partially differentiated cells, for example cells which are not functional T cells such as for example iPSC, mesoderm, HEC, HPC progenitor T cells or DP T cells, that is produced by a step in the methods described herein may be cultured, maintained or expanded before the next differentiation step. Partially differentiated cells may be expanded by any convenient technique.

After each step, the population of partially differentiated cells may contain 1% or more, 5% or more, 10% or more or 15% or more partially differentiated cells, following culture in the medium. If required, a population of partially differentiated cells may be purified by any convenient technique, such as MACs or FACS.

Cells may be cultured in a monolayer, in the absence of feeder cells, on a surface or substrate coated with extracellular matrix protein, such as fibronectin, laminin or collagen. Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% or 21% Oxygen, 5% Carbon Dioxide. Media is preferably changed every two days and cells allowed to settle by gravity.

Cells may be cultured in a culture vessel. Suitable cell culture vessels are well-known in the art and include culture plates, dishes, flasks, bioreactors, and multi-well plates, for example 6-well, 12-well or 96-well plates.

The culture vessels are preferably treated for tissue culture, for example by coating one or more surfaces of the vessel with an extracellular matrix protein, such as fibronectin, laminin or collagen. Culture vessels may be treated for tissue culture using standard techniques, for example by incubating with a coating solution, as described herein, or may be obtained from pre-treated from commercial suppliers.

In a first stage, iPSCs may be differentiated into mesoderm cells by culturing the population of iPSCs under suitable conditions to promote mesodermal differentiation. For example, the iPSCs cells may be cultured sequentially in first, second and third mesoderm induction media to induce differentiation into mesoderm cells.

A suitable first mesoderm induction medium may stimulate (i) SMAD2 and SMAD3 or (ii) SMAD2 and SMAD3 mediated signalling pathways. For example, the first mesoderm induction medium may comprise activin.

A suitable second mesoderm induction medium may (i) stimulate (a) SMAD1, SMAD2, SMAD3, SMAD5 and SMAD9 or (b) SMAD1, SMAD2, SMAD3, SMAD5 and SMAD9 mediated signalling pathways and (ii) have fibroblast growth factor (FGF) activity. For example, the second mesoderm induction medium may comprise activin, preferably activin A, BMP, preferably BMP4 and FGF, preferably bFGF.

A suitable third mesoderm induction medium may (i) stimulate (a) SMAD1, SMAD2, SMAD3, SMAD5 and SMAD9 or (b) SMAD1, SMAD2, SMAD3, SMAD5 and SMAD9 mediated signalling pathways (ii) have fibroblast growth factor (FGF) activity and (iii) inhibit glycogen synthase kinase 3β. For example, the third mesoderm induction medium may comprise activin, preferably activin A, BMP, preferably BMP4, FGF, preferably bFGF, and a GSK3 inhibitor, preferably CHIR99021.

The first, second and third mesoderm induction media may be devoid of differentiation factors other than the differentiation factors set out above.

SMAD2 and SMAD3 mediated intracellular signalling pathways may be stimulated by the first, second and third mesoderm induction media through the presence in the media of a first TGFβ ligand. The first TGFβ ligand may be Activin. Activin (Activin A: NCBI Gene ID: 3624 nucleic acid reference sequence NM_002192.2 GI: 62953137, amino acid reference sequence NP_002183.1 GI: 4504699) is a dimeric polypeptide which exerts a range of cellular effects via stimulation of the Activin/Nodal pathway (Vallier et al., Cell Science 118:4495-4509 (2005)). Activin is readily available from commercial sources (e.g. Stemgent Inc. MA USA; Miltenyi Biotec Gmbh, DE). Conveniently, the concentration of Activin in a medium described herein may be from 1 to 100 ng/ml, for example any of about 3, 5, 7, 9, 10, 15, 20, 25, 30, 35, 40, 45, 50 ng/ml, preferably about 5 to 50 ng/ml.

The fibroblast growth factor (FGF) activity of the second and third mesoderm induction media may be provided by the presence of fibroblast growth factor (FGF) in the media. Fibroblast growth factor (FGF) is a protein factor which stimulates cellular growth, proliferation and cellular differentiation by binding to a fibroblast growth factor receptor (FGFR). Suitable fibroblast growth factors include any member of the FGF family, for example any one of FGF1 to FGF14 and FGF15 to FGF23. Preferably, the FGF is FGF2 (also known as bFGF, NCBI GenelD: 2247, nucleic acid sequence NM_002006.3 GI: 41352694, amino acid sequence NP_001997.4 GI: 41352695); FGF7 (also known as keratinocyte growth factor (or KGF), NCBI GenelD: 2247, nucleic acid sequence NM_002006.3 GI: 41352694, amino acid sequence NP_001997.4 GI: 41352695); or FGF10 (NCBI GenelD: 2247, nucleic acid sequence NM_002006.3 GI: 41352694, amino acid sequence NP_001997.4 GI: 41352695). Most preferably, the fibroblast growth factor is FGF2.

Conveniently, the concentration of FGF, such as FGF2 in a medium described herein may be from 0.5 to 50 ng/ml, for example any of about 0.5, 1, 2, 3, 4, 5, 6, 7, 8, 9, 10, 11, 12, 13, 14, 15, 20, 25, 30, 35, 40, 45, 50 ng/ml, preferably about 5 ng/ml. Fibroblast growth factors, such as FGF2, FGF7 and FGF10, may be produced using routine recombinant techniques or obtained from commercial suppliers (e.g. R&D Systems, Minneapolis, Minn.; Stemgent Inc, USA; Miltenyi Biotec Gmbh, DE).

SMAD1, SMAD5 and SMAD9 mediated intracellular signalling pathways may be stimulated by the second and third mesoderm induction media through the presence in the media of a second TGFβ ligand. The second TGFβ ligand may be a Bone Morphogenic Protein (BMP). Bone Morphogenic Proteins (BMPs) bind to Bone Morphogenic Protein Receptors (BMPRs) and stimulate intracellular signalling through pathways mediated by SMAD1, SMAD5 and SMAD9. Suitable Bone Morphogenic Proteins include any member of the BMP family, for example BMP2, BMP3, BMP4, BMP5, BMP6 or BMP7. Preferably the second TGFβ ligand is BMP2 (NCBI GenelD: 650, nucleic acid sequence NM_001200.2 GI: 80861484; amino acid sequence NP_001191.1 GI: 4557369) or BMP4 (NCBI GenelD: 652, nucleic acid sequence NM_001202.3 GI: 157276592; amino acid sequence NP_001193.2 GI: 157276593). Suitable BMPs include BMP4. Conveniently, the concentration of a Bone Morphogenic Protein, such as BMP2 or BMP4 in a medium described herein may be from 1 to 500 ng/ml, for example any of about 0.5, 1, 2, 3, 4, 5, 6, 7, 8, 9, 10, 11, 12, 13, 14, 15, 20, 25, 30, 35, 40, 45, 50, 100, 150, 200, 250, 300, 350, 400, 450, 500 ng/ml, preferably about 10 ng/ml. BMPs may be produced using routine recombinant techniques or obtained from commercial suppliers (e.g. R&D, Minneapolis, USA, Stemgent Inc, USA; Miltenyi Biotec Gmbh, DE).

The GSK3β inhibition activity of the third mesoderm induction medium may be provided by the presence of a GSK3β inhibitor in the medium. GSK3β inhibitors inhibit the activity of glycogen synthase kinase 3β (Gene ID 2932: EC2.7.11.26). Preferred inhibitors specifically inhibit the activity of glycogen synthase kinase 3β. Suitable inhibitors include CHIR99021 (6-((2-((4-(2,4-Dichlorophenyl)-5-(4-methyl-1H-imidazol-2-yl)pyrimidin-2-yl)amino)ethyl)amino)nicotinonitrile; Ring D. B. et al., Diabetes, 52:588-595 (2003)) alsterpaullone, kenpaullone, BIO(6-bromoindirubin-3′-oxime (Sato et al Nat Med. 2004 January;10(1):55-63), SB216763 (3-(2,4-dichlorophenyl)-4-(1-methyl-1H-indol-3-yl)-1 H-pyrrole-2,5-dione), Lithium and SB415286 (3-[(3-chloro-4-hydroxyphenyl)amino]-4-(2-nitrophenyl)-1 H-pyrrole-2,5-dione; Coghlan et al Chem Biol. 2000 October;7(10):793-803). In some preferred embodiments, the GSK3β inhibitor is CHIR99021.Suitable glycogen synthase kinase 3β inhibitors may be obtained from commercial suppliers (e.g. Stemgent Inc. MA USA; Cayman Chemical Co. MI USA; Selleckchem, MA USA). For example, the third mesoderm induction medium may contain 0.1 to 100pM, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 μM, of a GSK3β inhibitor, such as CHIR99021, preferably about 10 μM.

In preferred embodiments, the first, second and third mesoderm induction media are chemically defined media. For example, the first mesoderm induction medium may consist of a chemically defined nutrient medium supplemented with an effective amount of activin, preferably activin A, for example 50 ng/ml activin A; the second mesoderm induction medium may consist of a chemically defined nutrient medium supplemented with an effective amount of activin preferably activin A, for example 5 ng/ml activin A, BMP, preferably BMP4, for example 10 ng/ml BMP4; and FGF, preferably bFGF (FGF2), for example 5 ng/ml bFGF; and the third mesoderm induction medium may consist of a chemically defined nutrient medium supplemented with an effective amount of activin preferably activin A, for example 5 ng/ml activin A, BMP, preferably BMP4, for example 10 ng/ml BMP4; FGF, preferably bFGF (FGF2), for example 5 ng/ml bFGF; and GSK3 inhibitor, preferably CHIR-99021 , for example 10 μM CHIR-99021.

A chemically defined medium (CDM) is a nutritive solution for culturing cells which contains only specified components, preferably components of known chemical structure. A CDM is devoid of undefined components or constituents which include undefined components, such as feeder cells, stromal cells, serum, and complex extracellular matrices, such as matrigel™. For example, a CDM does not contain stromal cells, such as OP9 cells, expressing Notch ligands, such as DLL1 or DLL4.

The chemically defined nutrient medium may comprise a chemically defined basal medium. Suitable chemically defined basal media include Iscove's Modified Dulbecco's Medium (IMDM), Ham's F12, Advanced Dulbecco's modified eagle medium (DMEM) (Price et al Focus (2003), 25 3-6), Williams E (Williams, G. M. et al Exp. Cell Research, 89, 139-142 (1974)), RPMI-1640 (Moore, G. E. and Woods L. K., (1976) Tissue Culture Association Manual. 3, 503-508) and StemPro™-34 PLUS (ThermoFisher Scientific).

The basal medium may be supplemented by serum-free culture medium supplements and/or additional components in the medium. Suitable supplements and additional components are described above and may include L-glutamine or substitutes, such as GlutaMAX-1™, ascorbic acid, monothiolglycerol (MTG), antibiotics such as penicillin and streptomycin, human serum albumin, for example recombinant human serum albumin, such as Cellastim™ (Merck/Sigma) and Recombumin™ (albumedix.com), insulin, transferrin, selenium (ITS) and 2-mercaptoethanol. A basal medium may be supplemented with a serum substitute, such as Knockout Serum Replacement (KOSR; Invitrogen).

The iPSCs may be cultured in the first mesoderm induction medium for 1 to 12 hours, for example any of 1, 2, 3, 4, 5, 6, 7, 8, 9, 10, 11 or 12 hours, preferably about 4 hours; then cultured in the second mesoderm induction medium for 30 to 54 hours, for example any of 35, 36, 37, 38, 39, 40, 41, 42, 43, 44, 45, 46, 47, 48, 49 or 50 hours preferably about 44 hours; and then cultured in the third mesoderm induction medium for 36 to 60 hours, for example any of 37, 38, 39, 40, 41, 42, 43, 44, 45, 46, 47, 48, 49, 50, 51, 52 or 53 hours preferably about 48 hours to produce a population of mesodermal cells.

Mesoderm cells are partially differentiated progenitor cells that are committed to mesodermal lineages and are capable of differentiation under appropriate conditions into all cell types in the mesenchyme (fibroblast), muscle, bone, adipose, vascular and haematopoietic systems. Mesoderm cells may express one or more mesodermal markers. For example, the mesoderm cells may express any one, two, three, four, five, six or all seven of Brachyury, Goosecoid, MixI1, KDR, FoxA2, GATA6 and PDGFαR.

In a second stage, mesoderm cells may be differentiated into haemogenic endothelial cells (HECs) by culturing the population of mesoderm cells under suitable conditions to promote haemogenic endothelial (HE) differentiation. For example, the mesoderm cells may be cultured in an HE induction medium.

A suitable HE induction medium may (i) stimulate cKIT receptor (CD117) or stimulate cKIT receptor (CD117) mediated signalling pathways and (ii) stimulate VEGFR or VEGFR mediated signalling pathways. For example, the HE induction medium may comprise SCF and VEGF.

SCF and VEGF are described above. Suitable HE induction media may include a haematopoietic induction medium described above.

In preferred embodiments, the HE induction medium is a chemically defined medium. For example, the HE induction medium may consist of a chemically defined nutrient medium supplemented with effective amounts of VEGF, for example 15 ng/ml VEGF; and SCF, for example 100 ng/ml SCF. A suitable HE induction medium may consist of a chemically defined nutrient medium supplemented with one or more differentiation factors, wherein the one or more differentiation factors consist of SCF and VEGF (i.e. the medium does not contain any differentiation factors other than SCF and VEGF).

Suitable chemically defined nutrient media are described above and include StemPro™-34 PLUS (ThermoFisher Scientific).

The mesoderm cells may be cultured in the HE induction medium for 2 to 6 days, for example any of 2, 3, 4, 5, or 6 days, preferably about 4 days, to produce a population of HECs.

The HECs may be differentiated into HPCs by culturing in a haematopoietic induction medium as described herein.

In some preferred embodiments, the same culture medium may be used as the HE induction medium and the haematopoietic induction medium. For example, mesoderm cells produced as described above may be differentiated into HPCs, such as HPCs and/or TPCs, using the same culture medium for both HE induction and haematopoietic induction.

Following the generation of HPCs from HECs as described herein, a population of HPCs expressing one or more cell surface markers, such as CD34, CD7 and/or CD45, may be purified, for example by magnetic activated cell sorting (MACS), before being subjected to further differentiation. For example, a population of CD34+ HPCs, CD34+CD45+ HPCs, CD34+CD7+ TPCs or CD34+CD45+ CD7+ TPCs may be purified.

The population of HPCs may comprise HPCs, thymocytic HPCs or both HPCs and thymocytic HPCs.

In a fourth stage, the HPCs may be differentiated into progenitor T cells by culturing the population of HPCs under suitable conditions to promote lymphoid differentiation. For example, the HPCs may be cultured in a lymphoid expansion medium.

A lymphoid expansion medium is a cell culture medium that promotes the lymphoid differentiation of HPCs into progenitor T cells.

A suitable lymphoid expansion medium may (i) stimulate cKIT receptor (CD117) or cKIT receptor (CD117) mediated signalling pathways, (ii) stimulate MPL (CD110) or MPL (CD110) mediated signalling pathways (iii) FLT3 or FLT3 mediated signalling pathways and (iv) have interleukin (IL) activity. For example, a lymphoid expansion medium may comprise the differentiation factors SCF, FLT3L, TPO and IL7.

In preferred embodiments, the lymphoid expansion medium is a chemically defined medium. For example, the lymphoid expansion medium may consist of a chemically defined nutrient medium supplemented with effective amounts of the above differentiation factors. Suitable lymphoid expansion media are well-known in the art and include Stemspan™ SFEM II (Cat # 9605; StemCell Technologies Inc, CA).with Stemspan™ lymphoid expansion supplement (Cat # 9915; StemCell Technologies Inc, CA).

The HPCs may be cultured on a surface during differentiation into progenitor T cells. For example, the HPCs may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers.

The surface may also be coated with an extracellular matrix protein, such as fibronectin, vitronectin, laminin or collagen and/or one or more cell surface adhesion proteins, such as VCAM1.

In some embodiments, the surface for HPC culture may have a coating that comprises a factor that stimulates Notch signalling, for example a Notch ligand, such as DLL4, an extracellular matrix protein, such as vitronectin, and a cell surface adhesion protein, such as VCAM1. In some embodiments, the surface for HPC culture may have a coating that comprises a factor that stimulates Notch signalling, for example a Notch ligand, such as DLL4, without the extracellular matrix protein or cell surface adhesion protein.

The surface may be coated with an extracellular matrix protein, factor that stimulates Notch signalling and cell surface adhesion protein by contacting the surface with a coating solution. For example, the coating solution may be incubated on the surface under suitable conditions to coat the surface. Conditions may, for example, include about 2 hours at room temperature. Coating solutions comprising an extracellular matrix protein and a factor that stimulates Notch signalling are available from commercial suppliers (StemSpan™ Lymphoid Differentiation Coating Material; Cat # 9925; Stem Cell Technologies Inc, CA).

The HPCs may be cultured in the lymphoid expansion medium on the substrate for a time sufficient for the HPCs to differentiate into progenitor T cells. For example, the HPCs may be cultured for 2-6 weeks, 2-5 weeks or 2-4 weeks, preferably 3 weeks.

Progenitor T cells are multi-potent lymphopoietic progenitor cells that are capable of giving rise to αβ T cells, γδ T cells, tissue resident T cells and NK T cells. Progenitor T cells may commit to the αβ T cell lineage after pre-TCR selection in the thymus. Progenitor T cells may be capable of in vivo thymus colonization and may be capable of committing to the αβ T cell lineage after pre-TCR selection in the thymus. Progenitor T cells may also be capable of maturation into cytokine-producing CD3+ T-cells.

Progenitor T cells may express CD5 and CD7 i.e. the progenitor T cells may have a CD5+CD7+ phenotype. Progenitor T cells may also co-express CD44, CD25 and CD2. For example, progenitor T cells may have a CD5+, CD7+ CD44+, CD25+ CD2+ phenotype. Progenitor T cells may also co-express CD45. Progenitor T cells may lack expression, for example cell surface expression, of CD3, CD4 and CD8.

In a fifth stage, progenitor T cells may be matured into double positive CD4+CD8+ T cells by culturing the population of progenitor T cells under suitable conditions to promote T cell maturation. For example, the progenitor T cells may be cultured in a T cell maturation medium.

A T cell maturation medium is a cell culture medium that promotes the maturation of progenitor T cells into mature T cells. A suitable T cell maturation medium may (i) stimulate cKIT receptor (CD117) or cKIT receptor (CD117) mediated signalling pathways (ii) stimulate FLT3 or FLT3 mediated signalling pathways and (iii) have interleukin (IL) activity. For example, a T cell maturation medium may comprise the differentiation factors SCF, FLT3L, and IL7.

In preferred embodiments, the T cell maturation medium is a chemically defined medium. For example, the T cell maturation medium may consist of a chemically defined nutrient medium supplemented with effective amounts of the above differentiation factors. Suitable T cell maturation media are well-known in the art and include Stemspan™ SFEM II (Cat # 9605; StemCell Technologies Inc, CA) with Stemspan™ T cell maturation supplement (Cat # 9930; StemCell Technologies Inc, CA) and other media suitable for expansion of PBMCs and CD3+ cells, such as ExCellerate Human T cell expansion medium (R& D Systems, USA). Other suitable T cell maturation media may include a basal medium such as IMDM, supplemented with ITS, albumin and lipids, as described elsewhere herein and further supplemented with effective amounts of the above differentiation factors.

The progenitor T cells may be cultured on a surface. For example, the progenitor T cells may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers. The surface may also be coated with an extracellular matrix protein, such as fibronectin, vitronectin, laminin or collagen and/or one or more cell surface adhesion proteins, such as VCAM1. Suitable coatings are well-known in the art and described elsewhere herein.

The progenitor T cells may be cultured in the T cell maturation medium on the substrate for a time sufficient for the progenitor T cells to mature into double positive CD4+CD8+ T cells. For example, the progenitor T cells may be cultured for 1-4 weeks, preferably 2 or 3 weeks.

T cells (also called T lymphocytes) are white blood cells that play a central role in cell-mediated immunity. T cells can be distinguished from other lymphocytes by the presence of a T cell receptor (TCR) on the cell surface. T cells express a T cell receptor (TCR).

There are several types of T cells, each type having a distinct function.

T helper cells (T_{H} cells) are known as CD4⁺T cells because they express the CD4 surface glycoprotein. CD4⁺ T cells play an important role in the adaptive immune system and help the activity of other immune cells by releasing T cell cytokines and helping to suppress or regulate immune responses. They are essential for the activation and growth of cytotoxic T cells. Cytotoxic T cells (Tc cells, CTLs, killer T cells, cytolytic T cells) are known as CD8⁺ T cells because they express the CD8 surface glycoprotein. CD8⁺ T cells act to destroy virus-infected cells and tumour cells. Most CD8⁺ T cells express TCRs that can recognise a specific antigen displayed on the surface of infected or damaged cells by a class I MHC molecule, or recognise a specific antigen displayed independent of MHC presentation, such T cells may be obtained according to the methods of the present invention. Specific binding of the TCR and CD8 glycoprotein to the antigen and MHC molecule leads to T cell-mediated destruction of the infected or damaged cells.

Double positive CD4+CD8+ T cells produced as described herein may be mature CD3+ T cells. In some embodiments, T cells may also express CD45 and CD28.

T cells produced as described herein may be γδ T cells, αβ T cells or NKT cells.

In some preferred embodiments, the T cells produced as described herein are βδ T cells. Following maturation of progenitor T cells (stage 5), the population of T cells may be predominantly double positive CD4+CD8+ T cells.

In a sixth stage, the population of double positive CD4+CD8+ T cells may be activated and/or expanded to produce or increase the proportion of single positive CD4+ T cells, or more preferably single positive CD8+ T cells.

Suitable methods for activating and expanding T cells are well-known in the art. For example, T cells may be exposed to a T cell receptor (TCR) agonist under appropriate culture conditions. Suitable TCR agonists include ligands, such as peptides displayed on a class I or II MHC molecule (MHC-peptide complexes) on the surface of a bead or an antigen presenting cell, such as a dendritic cell, and soluble factors, such as anti-TCR antibodies, for example anti-CD28 antibodies, and multimeric MHC-peptide complexes, such as MHC-peptide tetramers, pentamers or dextramers.

Activation refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA). In some embodiments, T cells may be activated by exposure to anti-αCD3 antibodies and IL2, IL7 or IL15. More preferably, T cells are activated by exposure to anti-αCD3 antibodies and anti-αCD28 antibodies. The activation may occur in the presence or absence of CD14⁺ monocytes. The T cells may be activated with anti-CD3 and anti-CD28 antibody coated beads. For example, PBMCs or T cell subsets including CD4⁺ and/or CD8⁺ cells may be activated, without feeder cells (antigen presenting cells) or antigen, using antibody coated beads, for example magnetic beads coated with anti-CD3 and anti-CD28 antibodies, such as Dynabeads® Human T-Activator CD3/CD28 (ThermoFisher Scientific). In other embodiments, soluble tetrameric antibody complexes that bind CD3, CD28 and CD2 cell surface ligands, such as ImmunoCult™ Human CD3/CD28/CD2 T Cell Activator or Human CD3/CD28 T Cell Activator, may be used to activate the T cells. In other embodiments, T cells may be activated with an MHC-peptide complex, preferably a multimeric MHC-peptide complex, optionally in combination with an anti-CD28 antibody.

T cells expressing a chimeric antigen receptor may be activated using a soluble antigen to the receptor. The antigen may be in a multimeric form or on the surface of a bead and may optionally be used in conjunction with an anti-TCR antibody, such as an anti-CD28 antibody.

In some embodiments, double positive CD4+CD8+ T cells may be cultured in a T cell maturation medium as described herein supplemented with IL-15. The medium may be further supplemented with a T cell receptor (TCR) agonist, for example one or more anti-TCR antibodies, such as anti-αCD3 antibodies, and anti-αCD28 antibodies, as described above.

T cells may be cultured using any convenient technique to produce the expanded population. Suitable culture systems include stirred tank fermenters, airlift fermenters, roller bottles, culture bags or dishes, and other bioreactors, in particular hollow fibre bioreactors. The use of such systems is well-known in the art

T cells produced as described herein may express an antigen receptor that binds a target antigen. For example, the antigen receptor may bind specifically to cancer cells that express a tumor antigen. The T cells may be useful for example in immunotherapy, as described below.

The antigen receptor may be a T cell receptor (TCR). TCRs are disulphide-linked membrane anchored heterodimeric proteins that comprise highly variable alpha (α) and beta (β) chains expressed as a complex with invariant CD3 chain molecules. T cells expressing this type of TCRs (αβ TCRs) may be referred to as αβ (or α:β) T cells. A minority of T cells express an alternative TCR comprising variable gamma (γ) and delta (δ) chains and are referred to as γδ T cells.

TCRs bind specifically to major histocompatibility complexes (MHC) on the surface of cells that display a peptide fragment of a target antigen. For example, TCRs may bind specifically to a major histocompatibility complex (MHC) on the surface of cancer cells that displays a peptide fragment of a tumour antigen. An MHC is a set of cell-surface proteins which allow the adaptive immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a cytotoxic effect on the cancer cell. In some embodiments the TCR may recognise target antigen or peptide fragment of target antigen on the cancer cell independently of MHC presentation.

In some embodiments, the TCR expressed by the T cells may be naturally expressed (i.e. an endogenous TCR). For example, the T cells may be produced as described herein from iPSCs that are derived from Tumour Infiltrating Lymphocytes (TILs). TILs, for example tumour resident CD3+ CD8+ cells, may be obtained from an individual with a cancer condition using standard techniques. Alternatively, the T cells may be produced as described herein from iPSCs that are derived from T cells that bind to a peptide fragment of the target antigen displayed on a class I or II MHC molecule on the surface of an antigen presenting cell, such as a dendritic cell; or a population of T cells produced as described herein may be screened for binding to a peptide fragment of the target antigen displayed on a class I or II MHC molecule, and T cells that bind to the displayed peptide fragment identified.

In other embodiments, the TCR is not naturally expressed by the cells (i.e. the TCR is exogenous or heterologous). Suitable heterologous αβ TCR may bind specifically to class I or II MHC molecules displaying peptide fragments of a target antigen. For example, the T cells may be modified to express a heterologous αγ TCR that binds specifically to class I or II MHC molecules displaying peptide fragments of a tumour antigen expressed by the cancer cells in a cancer patient. Tumour antigens expressed by cancer cells in the cancer patient may identified using standard techniques. In some embodiments the TCR may recognise target antigen or peptide fragment of target antigen on the cancer cell independently of MHC presentation. Preferred tumour antigens may include NY-ESO1, PRAME, alpha-fetoprotein (AFP), MAGE A4, MAGE A1, MAGE A10 and MAGE B2, most preferably NY-ESO-1, MAGE-A4 and MAGE-A10.

Suitable TCRs may include unconventional TCRs, for example non-MHC dependent TCRs that bind recognize non-peptide antigens displayed by monomorphic antigen-presenting molecules, such as CD1 and MR1; NKT cell TCRs and intraepithelial lymphocyte (IEL) TCRs. In some embodiments the TCR may recognise target antigen or peptide fragment of target antigen on the cancer cell independently of MHC presentation.

A heterologous TCR may be a synthetic or artificial TCR i.e. a TCR that does not exist in nature. For example, a heterologous TCR may be engineered to increase its affinity or avidity for a tumour antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains. These mutations increase the affinity of the TCR for MHCs that display a peptide fragment of a tumour antigen expressed by cancer cells. Suitable methods of generated affinity enhanced TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to cancer cells expressing one or more of the tumour antigens NY-ESO1, PRAME, alpha-fetoprotein (AFP), MAGE A4, MAGE A1, MAGE A10 and MAGE B2.

Alternatively, the antigen receptor may be a chimeric antigen receptor (CAR). CARs are artificial receptors that are engineered to contain an immunoglobulin antigen binding domain, such as a single-chain variable fragment (scFv). A CAR may, for example, comprise a scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J. S. et al. Proc Natl Acad Sci USA 1988; 85(16):5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 ζ-chain or from receptors such as CD28, 41 BB, or ICOS. A CAR may comprise multiple signalling domains, for 1example, but not limited to, CD3z-CD28-41 BB or CD3z-CD28-OX40.

The CAR may bind specifically to a tumour-specific antigen expressed by cancer cells. For example, the T cells may be modified to express a CAR that binds specifically to a tumour antigen that is expressed by the cancer cells in a specific cancer patient. Tumour antigens expressed by cancer cells in the cancer patient may identified using standard techniques.

Alternatively, the antigen receptor may be an NK cell receptor (NKCR).

Expression of a heterologous antigen receptor, such as a heterologous TCR, NKCR or CAR, may alter the immunogenic specificity of the T cells produced as described herein so that they recognise or display improved recognition for one or more target antigens, e.g. tumour antigens that are present on the surface of the cancer cells of an individual with cancer. In some embodiments, the T cells produced as described herein may display reduced binding or no binding to cancer cells in the absence of the heterologous antigen receptor. For example, expression of the heterologous antigen receptor may increase the affinity and/or specificity of the cancer cell binding of a T cell relative to T cells that do not express the antigen receptor.

The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. An endogenous polypeptide or nucleic acid is native to a particular biological system, such as a host cell, and is naturally present in that system. A recombinant polypeptide is expressed from heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

T cells may be modified to express the heterologous antigen receptor, such as a TCR or CAR, by the introduction of heterologous encoding nucleic acid into cells at any stage in the method described herein. For example, heterologous encoding nucleic acid may be introduced into iPSCs, mesoderm cells, HECs, HPCs, TPCs, or progenitor T cells. In some preferred embodiments, cells may be transduced with heterologous nucleic acid encoding the antigen receptor after 2 weeks culture in lymphoid expansion medium (stage 4) as described herein. Heterologous nucleic acid encoding an antigen receptor may encode all the sub-units of the receptor. For example, nucleic acid encoding a TCR may comprise a nucleotide sequence encoding a TCR α chain and a nucleotide sequence encoding a TCR β chain, or a nucleotide sequence encoding a TCR δ chain and a nucleotide sequence encoding a TCR γ chain.

Nucleic acid may be introduced into the cells by any convenient technique. When introducing or incorporating a heterologous nucleic acid into an iPSC, mesoderm cell, HEC, HPC, TPC or progenitor T cell, certain considerations must be taken into account, well-known to those skilled in the art. The nucleic acid to be inserted should be assembled within a construct or vector which contains effective regulatory elements which will drive transcription in the T cell. Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992. In some embodiments, nucleic acid may be introduced into the cells by gene editing. For example, a DNA double strand break (DSB) at a target site may be induced by a CRISPR/Cas9 system and the repair of the DSB may introduce the heterologous nucleic acid into the cell genome at the target site or the nucleic acid may be introducing using an rAAV vector (e.g. AAV mediated gene editing; Hirsch et al 2014 Methods Mol Biol 1114 291-307).

Suitable techniques for introducing the expression vector into the iPSCs, HPCs or progenitor T cells are well known in the art and include calcium phosphate transfection, DEAE-Dextran, electroporation, liposome-mediated transfection, gene editing and transduction using retrovirus or other virus, e.g. vaccinia or lentivirus. Preferably, nucleic acid encoding the heterologous TCR may be contained in a viral vector, most preferably a gamma retroviral vector or a lentiviral vector, such as a VSVg-pseudotyped lentiviral vector. A method described herein may comprise transducing a population of cells, for example iPSCs, HPCs or progenitor T cells, with a viral vector to produce a transduced population of genetically modified cells. The cells may be transduced by contact with a viral particle comprising the nucleic acid. Viral particles for transduction may be produced according to known methods. For example, HEK293T cells may be transfected with plasmids encoding viral packaging and envelope elements as well as a lentiviral vector comprising the coding nucleic acid. A VSVg-pseudotyped viral vector may be produced in combination with the viral envelope glycoprotein G of the Vesicular stomatitis virus (VSVg) to produce a pseudotyped virus particle. For example, solid-phase transduction may be performed without selection by culture on retronectin-coated, retroviral vector-preloaded tissue culture plates.

Following production, the population of T cells, for example double positive (DP) CD4+CD8+ cells, single positive (SP) CD4+ cells or single positive (SP) CD8+ cells, may be isolated and/or purified. Any convenient technique may be used, including fluorescence-activated cell sorting (FACS) or magnetic-activated cell sorting using antibody coated magnetic particles (MACS).

The population of T cells may be expanded and/or concentrated. Optionally, the population of T cells produced as described herein may be stored, for example or cryopreservation, before use.

A population of T cells may be admixed with other reagents, such as buffers, carriers, diluents, preservatives and/or pharmaceutically acceptable excipients. Suitable reagents are described in more detail below. A method described herein may comprise admixing the population of T cells with a pharmaceutically acceptable excipient.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the T cells, which may be DP CD4+CD8+ T cells, SP CD4+ T cells or preferably SP CD8+ T cells, may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

An aspect of the invention provides a population of T cells, which may be for example DP CD4+CD8+ T cells, SP CD4+ T cells or SP CD8+ T cells, produced by a method described above. The population of T cells may be for use as a medicament. For example, a population of mature T cells as described herein may be used in cancer immunotherapy therapy, for example adoptive T cell therapy.

Adoptive cellular therapy or adoptive immunotherapy refers to the adoptive transfer of human T lymphocytes that express antigen receptors that are specific for target cells. For example, human T lymphocytes may express TCRs that are specific for antigens expressed on target cells and/or that are specific for peptide MHC complexes expressed on target cells or chimeric antigen receptors (CAR) that are specific for antigens expressed on target cells.

This can be used to treat a range of diseases depending upon the target chosen, e.g., tumour specific antigens to treat cancer. Adoptive cellular therapy involves removing a portion of a donor's or the patient's cells, for example, white blood cells. The cells are then used to generate iPSCs in vitro and these iPSCs are used to efficiently generate T cells that are specific for antigens expressed on target cells and/or specific for peptide MHC complexes on target cells as described herein. The T cells may be expanded, washed, concentrated, and/or then frozen to allow time for testing, shipping and storage until a patient is ready to receive the infusion of cells.

Other aspects of the invention provide the use of a population of T cells produced as described herein for the manufacture of a medicament for the treatment of cancer, a population of T cells produced as described herein for the treatment of cancer, and a method of treatment of cancer comprising administering a population of T cells produced as described herein to an individual in need thereof.

The population of T cells may be autologous i.e. the T cells were originally obtained from the same individual to whom they are subsequently administered (i.e. the donor and recipient individual are the same).

The population of T cells may be allogeneic i.e. the T cells may be originally obtained from a different individual to the individual to whom they are subsequently administered (i.e. the donor and recipient individual are different). Allogeneic refers to a graft derived from a different animal of the same species.

The donor and recipient individuals may be HLA matched to avoid GVHD and other undesirable immune effects, such as rejection. Alternatively, the donor and recipient individuals may not be HLA matched, or HLA genes in the cells from the donor individual may be modified, for example by gene editing, to remove any HLA mismatch with the recipient.

A suitable population of T cells for administration to a recipient individual may be produced by a method comprising providing an initial population of cells obtained from a donor individual, reprogramming the cells into iPSCs and differentiating the iPSCs into T cells that express an antigen receptor, such as an αβ TCR, which binds specifically to cancer cells or antigen or peptide of antigen on cancer cells optionally presented in complex with MHC, in the recipient individual.

Following administration of the T cells, the recipient individual may exhibit a T cell mediated immune response against cancer cells in the recipient individual. This may have a beneficial effect on the cancer condition in the individual.

As used herein, the terms “cancer,” “neoplasm,” and “tumour” are used interchangeably and, in either the singular or plural form, refer to cells that have undergone a malignant transformation that makes them pathological to the host organism.

Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the TCR.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci AS et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumour effect is a biological effect which can be manifested by a reduction in the rate of tumour growth, decrease in tumour volume, a decrease in the number of tumour cells, a decrease in the number of metastases, an increase in life expectancy, or amelioration of various physiological symptoms associated with the cancerous condition. An “anti-tumour effect” can also be manifested by the ability of the peptides, polynucleotides, cells and antibodies, also T cells which may be obtained according to the methods of the present invention, as described herein in prevention of the occurrence of tumour in the first place

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of T cells, and a decrease in levels of tumour-specific antigens. Administration of T cells modified as described herein may improve the capacity of the individual to resist cancer growth, in particular growth of a cancer already present the subject and/or decrease the propensity for cancer growth in the individual.

The T cells or the pharmaceutical composition comprising the T cells may be administered to a subject by any convenient route of administration, whether systemically/ peripherally or at the site of desired action, including but not limited to; parenteral, for example, by infusion. Infusion involves the administration of the T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, for example any of about 1, 2, 3, 4, 5, 6, 7, 8, or 9, ×10⁵, ×10⁶, ×10⁷, ×10⁸, ×10⁹, or x 10¹⁰ cells per individual, typically 2×10⁸ to 2×10¹⁰ cells per individual, typically over the course of 30 minutes, with treatment repeated as necessary, for example at intervals of days to weeks. It will be appreciated that appropriate dosages of the TCR αβ+ T cells, and compositions comprising the TCR αβ+ T cells, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, cytokine release syndrome (CRS), the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

While the T cells may be administered alone, in some circumstances the T cells may be administered in combination with the target antigen, APCs displaying the target antigen, CD3/CD28 beads, IL-7, IL-2 and/or IL15 to promote expansion in vivo of the population of T cells. Administration in combination may be by separate, simultaneous or sequential administration of the combined components.

The population of T cells may be administered in combination with one or more other therapies, such as cytokines e.g. IL-2, cytotoxic chemotherapy, radiation and immuno-oncology agents, including checkpoint inhibitors, such as anti-B7-H3, anti-B7-H4, anti-TIM3, anti-KIR, anti-LAG3, anti-PD-1, anti-PD-L1, and anti-CTLA4 antibodies. Administration in combination may be by separate, simultaneous or sequential administration of the combined components.

The one or more other therapies may be administered by any convenient means, preferably at a site which is separate from the site of administration of the T cells.

Administration of T cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the T cells are administered in a single transfusion of any of 500 million, 1 billion, 2, 3, 4, 5, 6, 7, 8, 9, 10, 11, 12, 13, 14, 15 billion T cells for example at least 1×10⁹ T cells.

Other aspects of the invention provide kits and reagents for use in generating populations of HPCs, such as HPCs and TPCs, using the methods described above.

A kit for production of HPCs, such as HPCs and TPC may comprise;
a hematopoietic induction medium as described herein, for example a hematopoietic induction medium comprising VEGF and SCF; or a hematopoietic induction medium comprising VEGF, SCF, Thrombopoietin (TPO), Flt3 ligand (FIt3L), IGF-1, IL-3, IL-6, and optionally IL-7.

The kit may further comprise
a first mesoderm induction medium comprising activin,
a second mesoderm induction medium comprising activin, BMP and FGF, and/or
a third mesoderm induction medium comprising activin, BMP, FGF, and a GSK3 inhibitor.

Another aspect of the invention also provides the use of a set of culture media for the production of HPCs wherein the set of media comprises;
matopoietic induction medium comprising VEGF and SCF, for example a hematopoietic induction medium comprising VEGF, SCF, Thrombopoietin (TPO), Flt3 ligand (FIt3L), IGF-1, IL-3, IL-6, and optionally IL-7,
and optionally,
a first mesoderm induction medium comprising activin,
a second mesoderm induction medium comprising activin, BMP and FGF, and
a third mesoderm induction medium comprising activin, BMP, FGF, and a GSK3 inhibitor.

Suitable media are described in more detail above.

Media may be supplemented with effective amounts of the differentiation factors set out above, as described elsewhere herein.

The one or more culture media may be formulated in deionized, distilled water. The one or more media will typically be sterilized prior to use to prevent contamination, e.g. by ultraviolet light, heating, irradiation or filtration. The one or more media may be frozen (e.g. at −20° C. or −80° C.) for storage or transport. The one or more media may contain one or more antibiotics to prevent contamination.

The one or more media may be a lx formulation ora more concentrated formulation, e.g. a 2× to 250× concentrated medium formulation. In a 1× formulation each ingredient in the medium is at the concentration intended for cell culture, for example a concentration set out above. In a concentrated formulation one or more of the ingredients is present at a higher concentration than intended for cell culture. Concentrated culture media are well known in the art. Culture media can be concentrated using known methods e.g. salt precipitation or selective filtration. A concentrated medium may be diluted for use with water (preferably deionized and distilled) or any appropriate solution, e.g. an aqueous saline solution, an aqueous buffer or a culture medium.

The one or more media in the kit may be contained in hermetically-sealed vessels. Hermetically-sealed vessels may be preferred for transport or storage of the culture media, to prevent contamination. The vessel may be any suitable vessel, such as a flask, a plate, a bottle, a jar, a vial or a bag.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods disclosed herein. Concentrations described herein are determined at ambient temperature and pressure. This may be, for example, the temperature and pressure at room temperature or within a particular portion of a process stream.

Preferably, concentrations are determined at a standard state of 21 ° C. and 1 bar of pressure. The term “about” means a value within two standard deviations of the mean for any particular measured value.

As used herein and in the claims, the singular forms “a,” “and,” and “the” include plural reference unless the context clearly dictates otherwise. Thus, for example, reference to “a peptide chain” is a reference to one or more peptide chains and includes equivalents thereof known to those skilled in the art.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Experimental

hiPSC Culture

iPSCs were routinely cultured in mTeSR1 (SCT) on Matrigel (BD Corning) using tissue culture plasticware, in 5% CO₂, 5% O₂at 37° C. hiPSCs were harvested manually using an EasyPassage tool (Invitrogen) and cells seeded at 1:6 or 1:12 ratios, in media with Y27632 (R&D Systems) at 10 uM for the first 48h of culture. For differentiation, hiPSCs were passaged onto either matrigel or vitronectin at low density cultures using 1:48 or 1:98 split ratio. Seeding density was about 1 colony per field of view, when viewed under x4 magnification on the microscope, at 24h post seeding. hiPSCs were cultured in mTeSR1 or E8 flex (SCT), depending on the cell culture matrix used, for approx. 4-5 days until colonies were compacted and distinct cells were no longer visible.

T cell Differentiation from Pluripotent Stem Cells using Minimal (SV) Stage 3 medium 3 hiPSC cell lines, ChiPSC31, ADAP-J and NIH2, were maintained in hiPSC maintenance medium (mTeSR1 or E8 flex). The hiPSC maintenance medium was removed and the cells were washed twice with DMEM/F12. 2 mL of StemPro34 PLUS (StemPro34 from Invitrogen; StemPro34 basal media, with supplement added and Penicillin Streptomycin (1% v/v: Invitrogen) and Glutamine (2mM: Invitrogen), Ascorbic Acid (50 μg/ml: Sigma Aldrich) and monothioglycerol (100 μM: Sigma Aldrich), further supplemented with 50 ng/mL of Activin was added and incubated for 4 hours. Volumes are dependent on culture flask size, typically at least 2 mls/9 cm², and 20 mls/150 cm².

After 4 hours, the medium was removed and the cells were washed twice with DMEM/F12 to remove residual high concentration Activin A. The medium was replaced with 2 mL of StemPro34 PLUS supplemented with 5 ng/mL of Activin A, 10 ng/ml of BMP4 and 5 ng/ml of bFGF and incubated for 44 hours (Stage 1 media). The medium was then replaced with fresh Stage 1 media and supplemented with 10 μM CHIR-99021 and further cultured for 48 hours.

On Day 4, the medium was removed and the cells were washed twice with DMEM/F12 to remove residual stage 1 cytokines. The medium was then replaced with StemPro34 PLUS supplemented with 100 ng/mL of SCF and 15 ng/ml of VEGF and incubated for 48 hours (Stage 2 media). The medium was then replenished with fresh Stage 2 media and the cells cultured for a further 48 hours.

The medium was then replaced by the Stage 3 medium shown in Table 1 (HEM7) or a minimal medium comprising SCF and VEGF alone (SV). The cells were cultured for between 16-18 days, with 1:1 (v/v) feeding every 48h. Typically this involved harvest of media and collection of cells in suspension by centrifugation (at 300 g, 10 min), and returning suspension cells to culture with fresh media (i.e. 20 mls for a T150 flask).

On approx. d16-18 depending on hiPSC line used, (confirmed separately by flow cytometry prior to day of harvest) CD34+ or CD34+CD7+ cells were isolated from the resulting monolayers for onward culture. CD34+ cells were harvested by sequential incubation with Accutase (SCT: for 30 min at 37° C.) and then Collagenase II (Invitrogen: 2 mg/ml) for 30 min at 37° C. Cell suspensions were collected and washed (x2 centrifugation at 300 g for 12 min in DMEM/F12), prior to CD34+ cell isolation via Magnetic activated beads (MACS) isolation (Miltenyi: according to manufacturer's instructions).

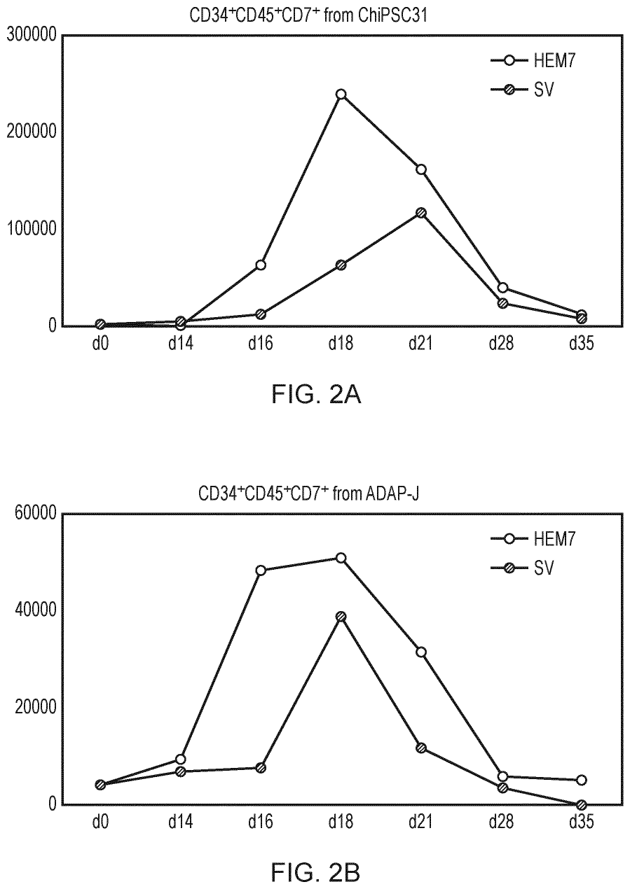

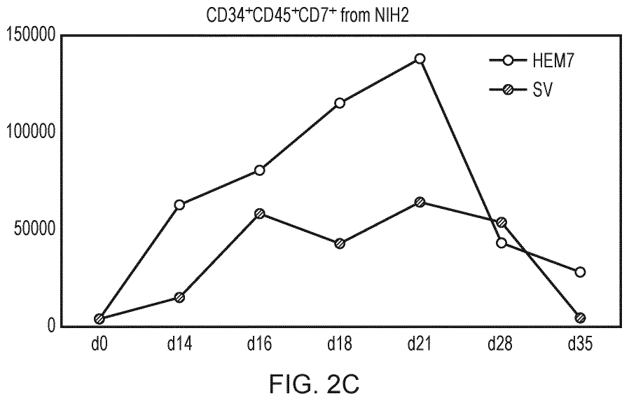

Upon subsequent isolation of cells from the monolayer at defined time-points d12-d21, also as described with Accutase and Collagenase, cells were stained for flow cytometry using appropriate antibodies (CD34, CD45, and CD7). Emergence of CD34+CD45+ CD7+ progenitor cells were compared between standard Stage 3 media (HEM7) and media with SCF and VEGF alone (SV), as the minimal option for Stage 3 media (FIG. 2).

CD34+CD45+ CD7+ cells were found to emerge under full (HEM7) and minimal (SV) Stage 3 conditions from d16-d28 (FIG. 2) with 3 separate iPSC lines tested (ChiPSC31, ADAP-J, NIH2).

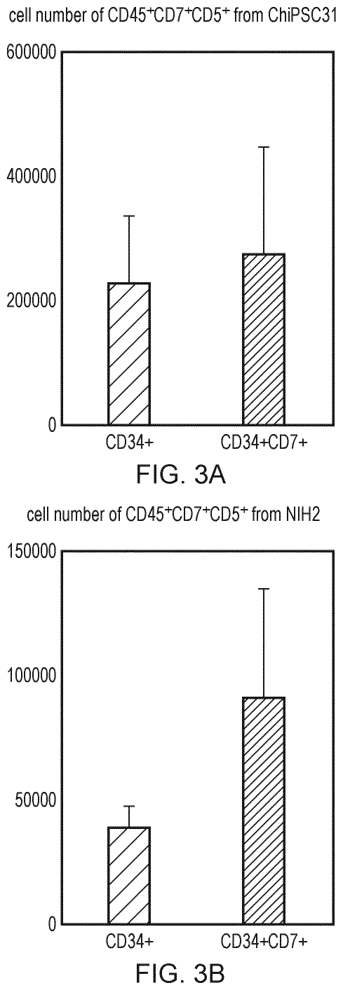

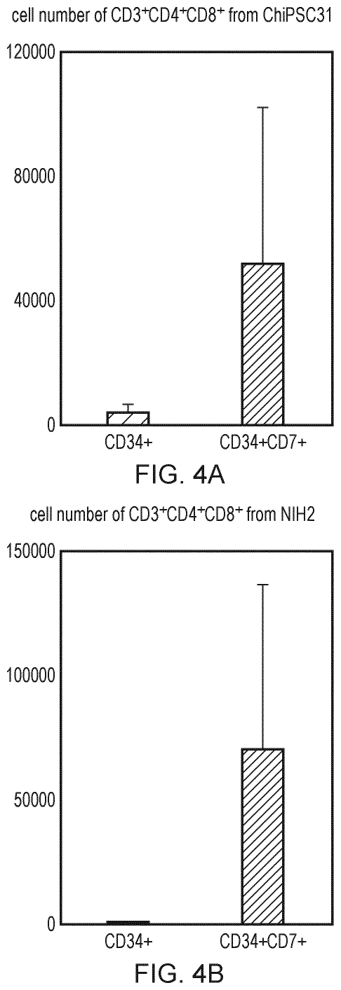

Upon subsequent isolation of cells from the monolayer on d16-18, CD34+ HPCs and CD34+ CD7+ cells from ChiPSC31 and NIH2 hiPSC lines were assessed for their ability to contribute proT cells (CD7+ CD5+) after 14 days culture in SCT LP media (FIG. 3), and at 21 days where CD4+ CD8+ CD3+ expression was assessed (FIG. 4) after appropriate staining and flow cytometry.

It was found that CD34+ or CD34+ CD7+ cells produced from hiPSCs using a haematopoietic induction medium described herein (“minimal Stage 3 media”) were capable of generating CD7+ CD5+ proT cells when further cultured in lymphoid proliferation (LP) media (Stem Cell Technologies) for a further 14 days.

It was also found that CD34+ or CD34+ CD7+ cells produced from hiPSCs using a haematopoietic induction medium described herein (“minimal Stage 3 media”) were capable of generating CD3+, CD4+ and CD8+ double positive T cells when further cultured in LP media for a further 21 days.

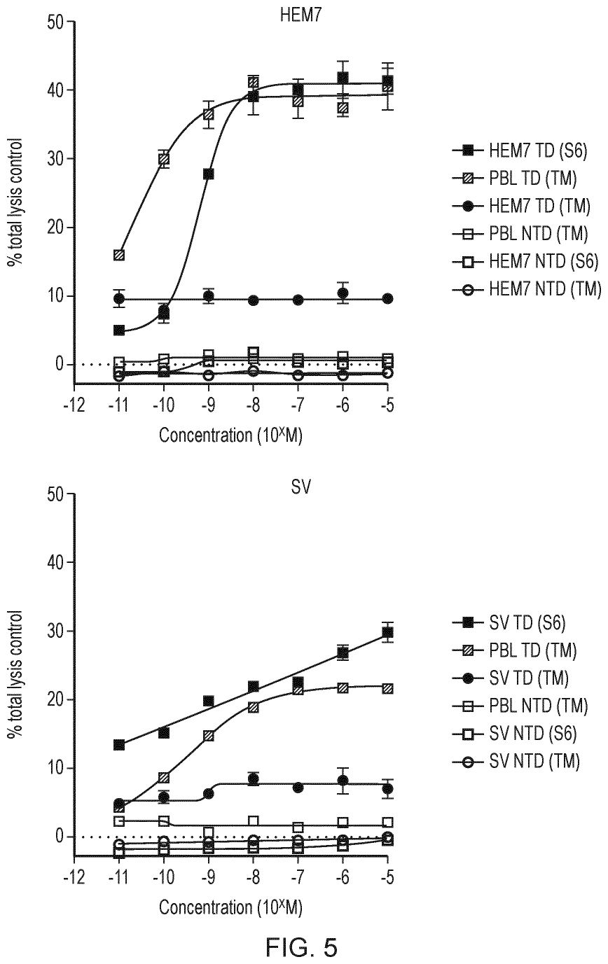

CD34+ HPCs from both full (HEM7) and minimal (SV) conditions were cultured in SCT medium for T cell proliferation (LP) media and then transduced with a lentiviral construct expressing a heterologous TCR which specifically recognises and binds an antigen MAGE-A4 derived peptide fragment in combination with HLA-A2. The derived T cells were then cultured in T cell maturation (TM) media and further stage 6 medium with activators, as described herein. The mature T cells were then evaluated in a KILRTM assay. Antigen-specific killing was observed by T cells generated using both full (HEM7) and minimal (SV) Stage 3 conditions (FIG. 5).

T cell Differentiation from Pluripotent Stem Cells using Modified (HEM7.2) Stage 3 Medium

hiPSC cell line NIH2, was maintained in hiPSC maintenance medium (mTeSR1 or E8 flex). The hiPSC maintenance medium was removed and the cells were washed twice with DMEM/F12. 2 mL of StemPro34 PLUS (StemPro34 from Invitrogen; StemPro34 basal media, with supplement added and Penicillin Streptomycin (1% v/v: Invitrogen) and Glutamine (2 mM: Invitrogen), Ascorbic Acid (50 μg/ml: Sigma Aldrich) and monothioglycerol (100 μM: Sigma Aldrich), further supplemented with 50 ng/mL of Activin was added and incubated for 4 hours. Volumes are dependent on culture flask size, typically at least 2 mls/9 cm², and 20 mls /150 cm².

After 4 hours, the medium was removed and the cells were washed twice with DMEM/F12 to remove residual high concentration Activin A. The medium was replaced with 2 mL of StemPro34 PLUS supplemented with 5 ng/mL of Activin A, 10 ng/ml of BMP4 and 5 ng/ml of bFGF and incubated for 44 hours (Stage 1 media). The medium was then replaced with fresh Stage 1 media and supplemented with 10 μM CHIR-99021 and further cultured for 48 hours.

On Day 4, the medium was removed and the cells were washed twice with DMEM/F12 to remove residual stage 1 cytokines. The medium was then replaced with StemPro34 PLUS supplemented with 100 ng/mL of SCF and 15 ng/ml of VEGF and incubated for 48 hours (Stage 2 media). The medium was then replenished with fresh Stage 2 media and the cells cultured for a further 48 hours.

The medium was then replaced by the Stage 3 medium shown in Table 1 (HEM7) or a modified Stage 3 medium comprising VEGF, SCF, Thrombopoietin (TPO), Flt3 ligand (FIt3L), IL-3, IL-6, IGF-1 only (HEM7.2). The cells were cultured for between 16-18 days, with 1:1 (v/v) feeding every 48h. Typically this involved harvest of media and collection of cells in suspension by centrifugation (at 300 g, 10 min), and returning suspension cells to culture with fresh media (i.e. 20m1s for a T150 flask).

On approx. d16, CD34+ or CD34+CD7+ cells were isolated from the resulting monolayers for onward culture. CD34+ cells were harvested by sequential incubation with Accutase (SCT: for 30 min at 37° C.) and then Collagenase II (Invitrogen: 2 mg/ml) for 30 min at 37° C. Cell suspensions were collected and washed (×2 centrifugation at 300 g for 12 min in DMEM/F12), prior to CD34+ cell isolation via Magnetic activated beads (MACS) isolation (Miltenyi: according to manufacturer's instructions). Cells were stained for flow cytometry using appropriate fluorescent antibodies (CD34, CD45, and CD7).

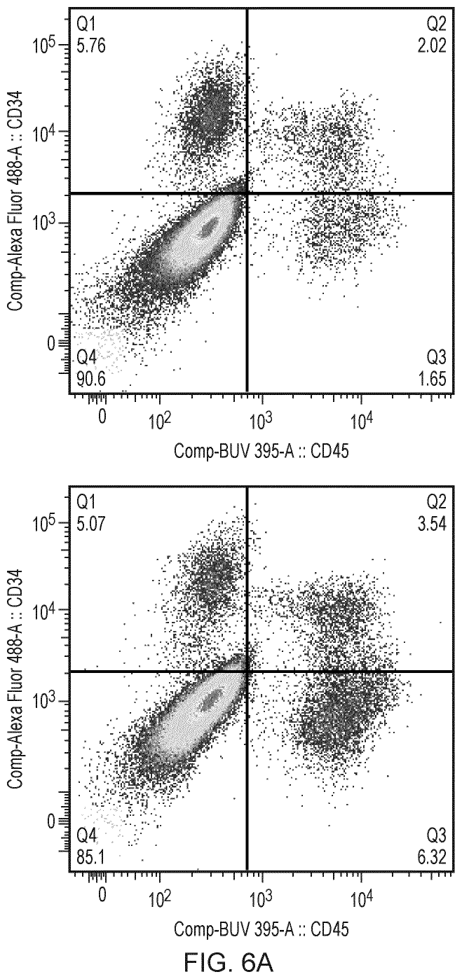

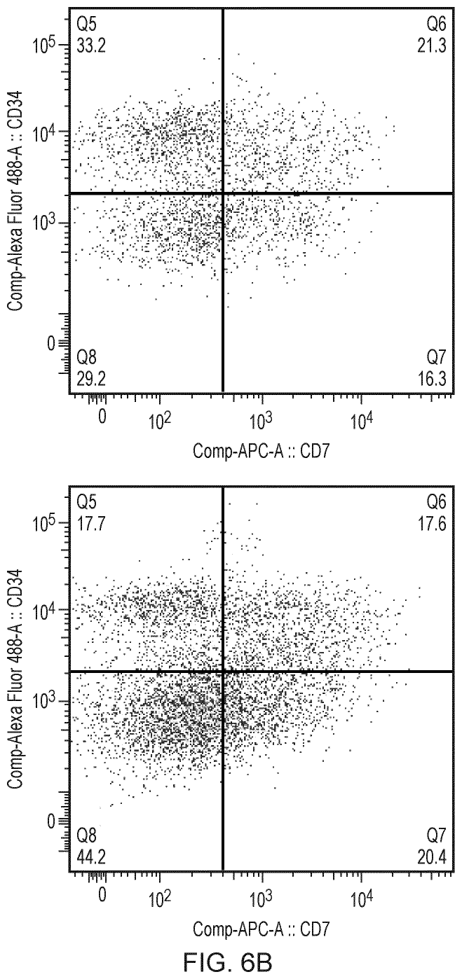

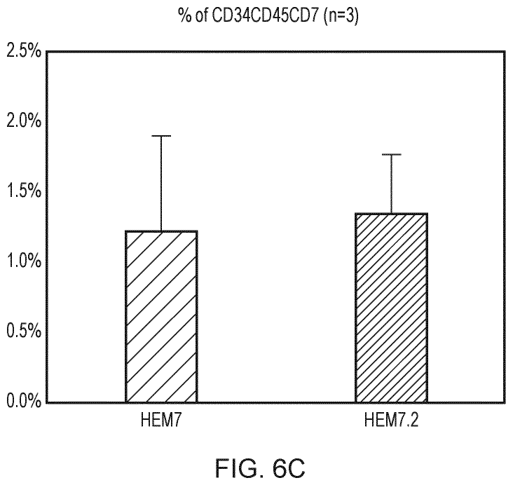

The emergence of CD34+ CD45+ CD7+ progenitor cells was compared between standard Stage 3 medium (HEM7) and modified Stage 3 medium (HEM7.2) (FIG. 6). CD34+ CD45+ CD7+ cells were found to emerge under both standard (HEM7) and modified (HEM7.2) Stage 3 conditions on d16 (FIG. 6).

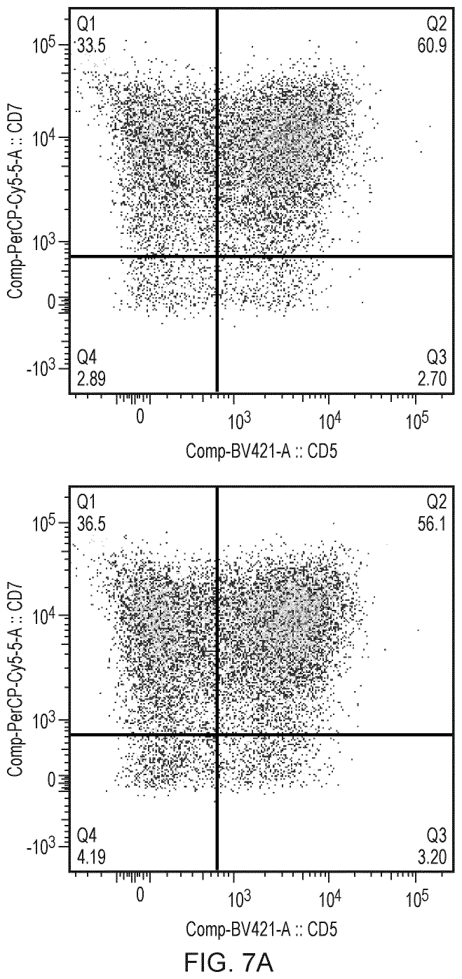

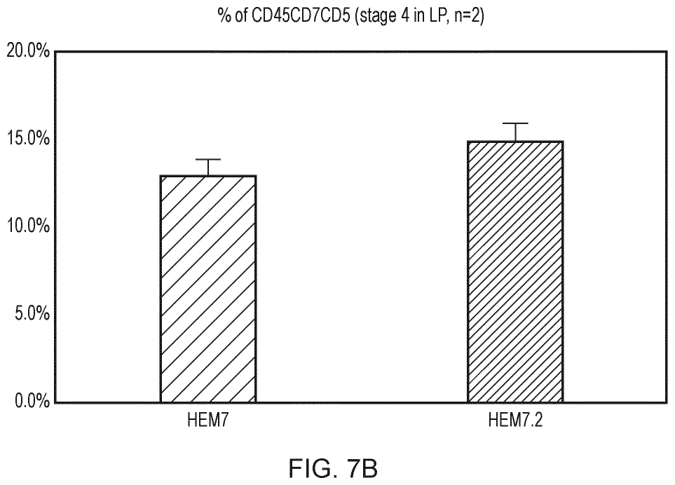

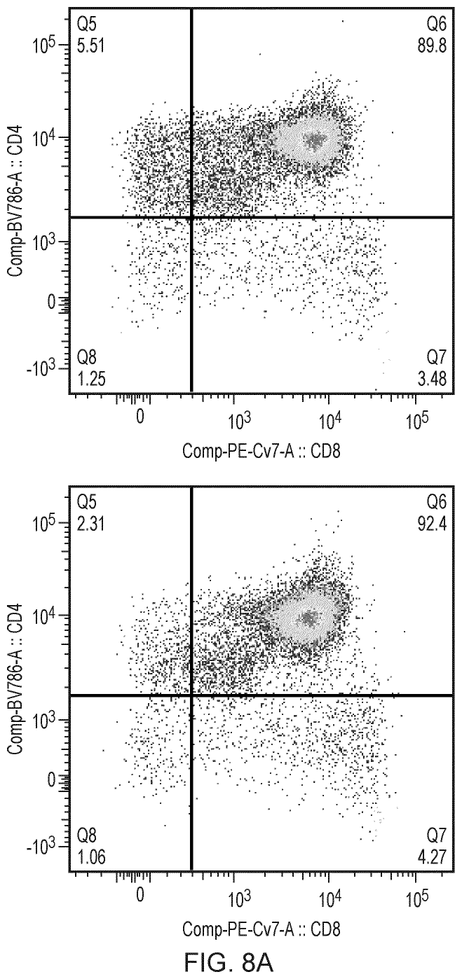

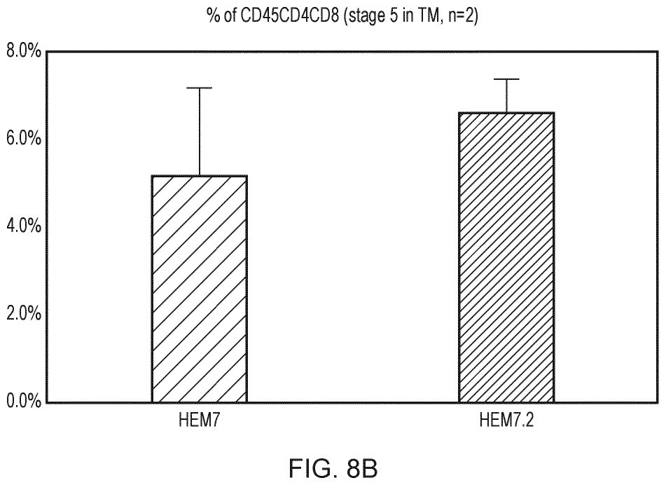

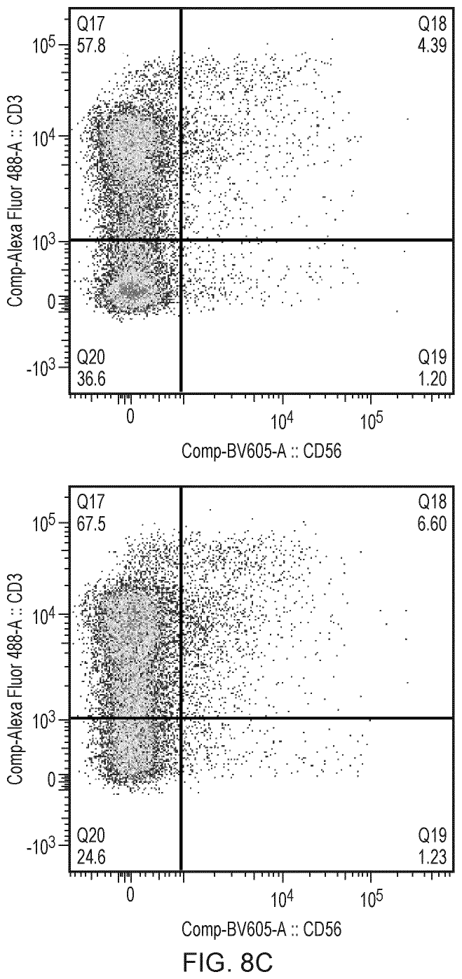

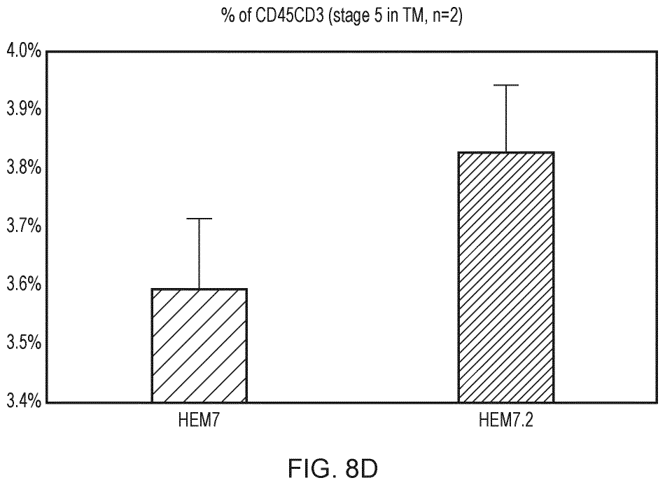

Upon subsequent isolation of cells from the monolayer on d16, CD34+ HPCs from NIH2 hiPSC lines were assessed for their ability to contribute proT cells (CD7+ CD5+) after 21 days culture in SCT LP medium (FIG. 7), and further 21 days in SCTs TM medium, where CD4+ CD8+ CD3+ expression was assessed (FIG. 8) after appropriate staining and flow cytometry.

It was found that CD34+ cells produced from hiPSCs using a haematopoietic induction medium described herein (“modified Stage 3 medium”) were capable of generating CD7+ CD5+ proT cells when further cultured in lymphoid proliferation (LP) media (Stem Cell Technologies) for a further 14 days.

It was also found that CD34+ cells produced from hiPSCs using a haematopoietic induction medium described herein (“modified Stage 3 medium”) were capable of generating CD3+, CD4+ and CD8+ double positive T cells when further cultured in LP media for a further 21 days.

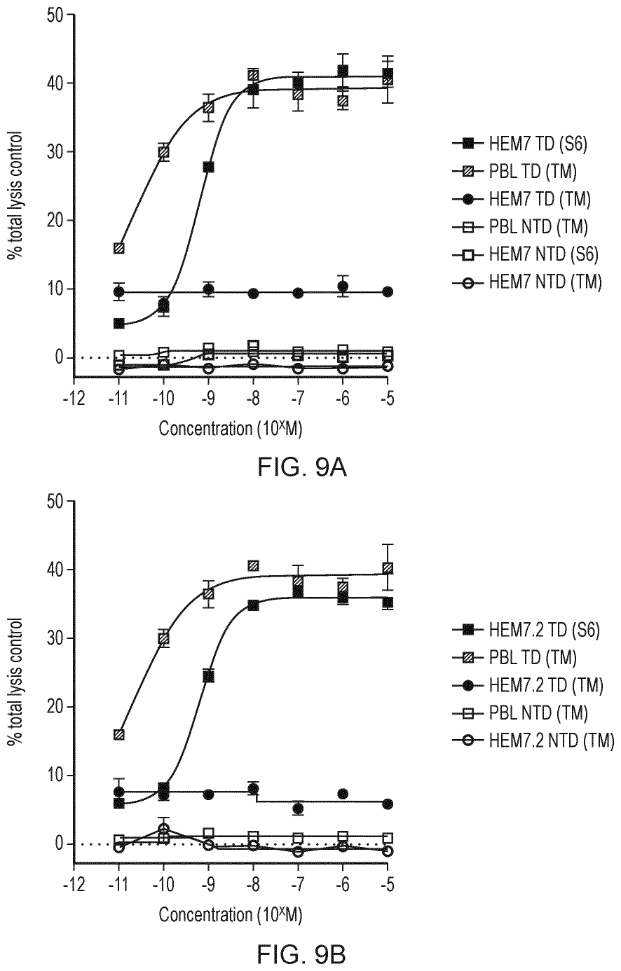

CD34+ HPCs were isolated from both standard (HEM7) and new (HEM7.2) Stage 3 conditions on day 16. Typically cells were cultured in SCT proprietary media for T cell proliferation (LP) media and then transduced with MAGE-A4 (TD) or Non transduction (NTD). The derived T cell were then cultured in T cell maturation (TM) media and further stage 6 media with activators (S6). Mature T cells were evaluated in a KILR assay. Obvious antigen-specific killing by T cells generated from both standard (HEM7) and modified (HEM7.2) Stage 3 conditions was observed (FIG. 9)

T cell Differentiation from Pluripotent Stem Cells using Modified (HEM7.3) Stage 3 Medium

hiPSC cell lines, were maintained in hiPSC maintenance medium (mTeSR1 or E8 flex). The hiPSC maintenance medium was removed and the cells were washed twice with DMEM/F12. 2 mL of StemPro34 PLUS (StemPro34 from Invitrogen; StemPro34 basal media, with supplement added and Penicillin Streptomycin (1% v/v: Invitrogen) and Glutamine (2 mM: Invitrogen), Ascorbic Acid (50 μg/ml: Sigma Aldrich) and monothioglycerol (100 μM: Sigma Aldrich), further supplemented with 50 ng/mL of Activin was added and incubated for 4 hours. Volumes are dependent on culture flask size, typically at least 2 mls/9 cm², and 20 mIs/150 cm².

After 4 hours, the medium was removed and the cells were washed twice with DMEM/F12 to remove residual high concentration Activin A. The medium was replaced with 2 mL of StemPro34 PLUS supplemented with 5 ng/mL of Activin A, 10 ng/ml of BMP4 and 5 ng/ml of bFGF and incubated for 44 hours (Stage 1 media). The medium was then replaced with fresh Stage 1 media and supplemented with 10 μM CHIR-99021 and further cultured for 48 hours.

On Day 4, the medium was removed and the cells were washed twice with DMEM/F12 to remove residual stage 1 cytokines. The medium was then replaced with StemPro34 PLUS supplemented with 100 ng/mL of SCF and 15 ng/ml of VEGF and incubated for 48 hours (Stage 2 media). The medium was then replenished with fresh Stage 2 media and the cells cultured for a further 48 hours.

The medium was then replaced by a modified Stage 3 medium comprising VEGF, SCF, Thrombopoietin (TPO), Flt3 ligand (FIt3L), IL-3, IL-6, IGF-1 and IL-7 only (HEM7.3) or HEM7.2. The cells were cultured for between 16-18 days, with 1:1 (v/v) feeding every 48h. Typically this involved harvest of media and collection of cells in suspension by centrifugation (at 300 g, 10 min), and returning suspension cells to culture with fresh media (i.e. 20m1s for a T150 flask).

On approx. d16, CD34+ or CD34+CD7+ cells were isolated from the resulting monolayers for onward culture. CD34+ cells were harvested by sequential incubation with Accutase (SCT: for 30 min at 37° C.) and then Collagenase II (Invitrogen: 2 mg/ml) for 30 min at 37° C. Cell suspensions were collected and washed (×2 centrifugation at 300 g for 12 min in DMEM/F12), prior to CD34+ cell isolation via Magnetic activated beads (MACS) isolation (Miltenyi: according to manufacturer's instructions). Cells were stained for flow cytometry using appropriate fluorescent antibodies (CD34, CD45, and CD7).

The emergence of CD34+ CD45+ CD7+ progenitor cells was assessed using modified Stage 3 medium (HEM7.3). CD34⁺ CD45⁺ CD7+ cells were found to emerge under modified (HEM7.3) Stage 3 conditions on d16.

Upon subsequent isolation of cells from the monolayer on d16, CD34+ HPCs from the hiPSC lines were assessed for their ability to contribute proT cells (CD7+ CD5+) after 21 days culture in SCT LP medium, and further 21 days in SCTs TM medium, where CD4+CD8+CD3+expression was assessed after appropriate staining and flow cytometry.

TABLE 1
(Hem7 medium)

| Reagent | Final Concentration | |
|---|---|---|
| StemPro34 PLUS | 1 L (2 bottles required) | |
| VEGF | 15 | ng/mL |
| SCF | 100 | ng/mL |
| TPO | 30 | ng/mL |
| Flt3L | 25 | ng/mL |
| IL-3 | 25 | ng/mL |
| IL-6 | 10 | ng/mL |
| IL-7 | 10 | ng/mL |
| IL-11 | 5 | ng/mL |
| IGF-1 | 25 | ng/mL |
| BMP-4 | 10 | ng/mL |
| bFGF | 5 | ng/mL |
| SHH | 25 | ng/mL |
| EPO | 2 | U/mL |
| Angiotensin II | 10 | μg/mL |
| Losartan | 100 | μM |

TABLE 2
Antibodies used to phenotype T cells.

| Antibody | Volume/test (μl) |
|---|---|
| TCRαβ (IP26) PE (BioLegend: 306708) | 2.5 μl |
| TCRγδ (B1) APC (BioLegend: 331212) | 5 μl |
| CD5 (UCHT2) BV421 (BD: 562646) | 5 μl |
| CD7 (CD7-6B7) PerCP Cy5.5 (BioLegend: 343116) | 5 μl |
| CD45 (HI30) BUV395 (BD: 563792) | 5 μl |
| CD4 (OKT4) BV786 (BioLegend: 317442) | 5 μl |
| CD3 (SK7) AF488 (BioLegend: 344810) | 5 μl |
| CD8α (RPA-T8) PE-Cy7 (BD: 557746) | 5 μl |
| CD56 (NCAM16.2) BV605 (BD: 562780) | 5 μl |
| Ef506 BV510 (Invitrogen: 65-0866-14) | 1/100 dilution |

TABLE 3
(HEM7.2 medium)

| | |
|---|---|
| StemPro34 PLUS | 1 L (2 bottles required) |
| VEGF | 15 ng/mL |
| SCF | 100 ng/mL |
| TPO | 30 ng/mL |
| Flt3L | 25 ng/mL |
| IL-3 | 25 ng/mL |
| IL-6 | 10 ng/mL |
| IGF-1 | 25 ng/mL |

TABLE 4
(HEM7.3 medium)

| | |
|---|---|
| StemPro34 PLUS | 1 L (2 bottles required) |
| VEGF | 15 ng/mL |
| SCF | 100 ng/mL |
| TPO | 30 ng/mL |
| Flt3L | 25 ng/mL |
| IL-3 | 25 ng/mL |
| IL-6 | 10 ng/mL |
| IL-7 | 10 ng/mL |
| IGF-1 | 25 ng/mL |

1. A method of producing a population of haematopoietic progenitor cells (HPCs) comprising;
(i) culturing a population of haemogenic endothelial cells (HECs) in a haematopoietic induction medium to produce a population of haematopoietic progenitor cells (HPCs),
wherein the haematopoietic induction medium (i) stimulates cKIT receptor (CD117) or cKIT receptor (CD117) mediated signalling pathways and/or (ii) stimulates VEGFR or VEGFR mediated signalling pathways.

2. A method according to claim 1 wherein the haematopoietic induction medium comprises one or both of SCF and VEGF.

3. A method according to claim 2 wherein the haematopoietic induction medium comprises SCF and VEGF.

4. A method according to any one of claims 1 to 3 wherein the haematopoietic induction medium (iii) stimulates MPL (CD110) or MPL (CD110) mediated signalling pathways; (iv) stimulates FLT3 or FLT3 mediated signalling pathways (v) stimulates IGF1R or IGF1R mediated signalling pathways and (vi) displays interleukin (IL) activity.

5. A method according to claim 4 wherein the haematopoietic induction medium comprises VEGF, SCF, Thrombopoietin (TPO), Flt3 ligand (FIt3L), IGF-1, IL-3 and IL-6.

6. A method according to claim 4 wherein the haematopoietic induction medium comprises VEGF, SCF, Thrombopoietin (TPO), Flt3 ligand (FIt3L), IGF-1, IL-3, IL-6, and IL-7.

7. A method according to any one of claims 1 to 6 wherein the haematopoietic induction medium is devoid of one or more of BMP, FGF, SHH, EPO, angiotensin II and losartan.

8. A method according to any one of claims 1 to 3 wherein the haematopoietic induction medium consists of a chemically defined nutrient medium supplemented with one or more differentiation factors, wherein the one or more differentiation factors consist of SCF and/or VEGF.

9. A method according to any one of claims 1 to 7 wherein the haematopoietic induction medium consists of a chemically defined nutrient medium supplemented with one or more differentiation factors, wherein (i) the one or more differentiation factors consist of VEGF, SCF, Thrombopoietin (TPO), Flt3 ligand (FIt3L), IGF-1, IL-3 and IL-6; or (ii) the one or more differentiation factors consist of VEGF, SCF, Thrombopoietin (TPO), Flt3 ligand (FIt3L), IGF-1, IL-3, IL-6, and IL-7.

10. A method according to claim 9 wherein the differentiation factors in the haematopoietic induction medium are (i) VEGF, SCF, Thrombopoietin (TPO), FIt3 ligand (FIt3L), IGF-1, IL-3, IL-6, and IL-7; or (ii) VEGF, SCF, Thrombopoietin (TPO), FIt3 ligand (FIt3L), IGF-1, IL-3, and IL-6.

11. A method according to any one of claims 1 to 10 wherein the HECs display a CD34+CD73-CXCR4-phenotype.

12. A method according to claim 11 wherein the HPCs display a CD34+ phenotype.

13. A method according to any one of the preceding claims wherein the HPCs comprise thymopoietic HPCs (tHPCs).

14. A method according to claim 13 wherein the tHPCs display a CD34+ CD7+phenotype

15. A method according to any one of the preceding claims wherein the HECs are cultured in the haematopoietic induction medium for 16-28 days to produce the HPCs.

16. A method according to any one of the preceding claims wherein the population of HECs is produced in vitro from induced pluripotent stem cells (iPSCs).

17. A method according to claim 16 wherein the method comprises
(i) differentiating a population of induced pluripotent stem cells (iPSCs) into mesoderm cells; and
(ii) differentiating the mesoderm cells to produce a population of haemogenic endothelial cells (HECs).

18. A method according to claim 16 or claim 17 wherein the iPSCs are derived from T cells obtained from a donor individual.

19. A method according to claim 18 wherein the T cells obtained from the donor individual are specific for a target antigen.

20. A method according to claim 19 wherein the target antigen is a tumour antigen.

21. A method according to claim 19 or 20 wherein the T cells obtained from the donor individual are tumour-infiltrating lymphocytes (TILs).

22. A method according to any one of claims 17 to 21 wherein the iPSCs are differentiated into mesoderm cells by culturing the population of iPSCs under suitable conditions to promote mesodermal differentiation.

23. A method according to any one of claims 17 to 22 wherein the iPSCs are cultured sequentially in first, second and third mesoderm induction media to induce differentiation into mesoderm cells.

24. A method according to claim 23 wherein the first mesoderm induction medium stimulates SMAD2 and SMAD3 or SMAD2 and SMAD3 mediated signalling pathways.

25. A method according to claim 24 wherein the first mesoderm induction medium comprises activin.

26. A method according to claim 24 or claim 25 wherein the first mesoderm induction medium consists of a chemically defined nutrient medium supplemented with one or more differentiation factors, wherein the one or more differentiation factors consist of activin.

27. A method according to any one of claims 23 to 26 wherein the second mesoderm induction medium (i) stimulates (a) SMAD1, SMAD2, SMAD3, SMAD5 and SMAD9 or (b) SMAD1, SMAD2, SMAD3, SMAD5 and SMAD9 mediated signalling pathways and (ii) has fibroblast growth factor (FGF) activity.

28. A method according to claim 27 wherein the second mesoderm induction medium comprises activin, BMP, and FGF.

29. A method according to claim 27 or claim 28 wherein the second mesoderm induction medium consists of a chemically defined nutrient medium supplemented with one or more differentiation factors, wherein the one or more differentiation factors consist of activin, BMP, and FGF.

30. A method according to any one of claims 23 to 29 wherein the third mesoderm induction medium (i) stimulates (a) SMAD1, SMAD2, SMAD3, SMAD5 and SMAD9 or (b) SMAD1, SMAD2, SMAD3, SMAD5 and SMAD9 mediated signalling pathways (ii) has fibroblast growth factor (FGF) activity and (iii) inhibits glycogen synthase kinase 3β.

31. A method according to claim 30 wherein the third mesoderm induction medium comprises activin, BMP, FGF, and a GSK3 inhibitor.

32. A method according to claim 31 wherein the third mesoderm induction medium consists of a chemically defined nutrient medium supplemented with one or more differentiation factors, wherein the one or more differentiation factors consist of activin, BMP, FGF, and a GSK3 inhibitor.

33. A method according to any one of claims 23 to 32 wherein the mesoderm cells display one or more of Brachyury, Goosecoid, MixI1, KDR, FoxA2, GATA6 and PDGFαR .

34. A method according to any one of claims 23 to 33 wherein the mesoderm cells are differentiated into HECs by culturing the population of mesoderm cells under suitable conditions to promote haemogenic endothelial (HE) differentiation.

35. A method according to any one of claims 23 to 34 wherein the mesoderm cells are cultured in an HE induction medium to induce differentiation into HECs.

36. A method according to claim 35 wherein the HE induction medium (i) stimulates cKIT receptor (CD117) or cKIT receptor (CD117) mediated signalling pathways and (ii) stimulates VEGFR or VEGFR mediated signalling pathways.

37. A method according to claim 36 wherein the HE induction medium comprises SCF and VEGF.

38. A method according to claim 37 wherein the HE induction medium consists of a chemically defined nutrient medium supplemented with one or more differentiation factors, wherein the one or more differentiation factors consist of SCF and VEGF.

39. A method according to any one of the preceding claims further comprising differentiating the population of HPCs into progenitor T cells.

40. A method according to claim 39 wherein the HPCs are differentiated by a method comprising culturing the population of HPCs in a lymphoid expansion medium to produce the progenitor T cells.

41. A method according to claim 39 or 40 wherein the progenitor T cells have a CD5+CD7+phenotype.

42. A method according to any one of claims 39 to 41 further comprising maturing the progenitor T cells to produce a population of double positive CD8+ CD4+ T cells.

43. A method according to claim 42 wherein the progenitor T cells are matured by a method comprising culturing the population of progenitor T cells in a T cell maturation medium to produce the double positive CD8+ CD4+ T cells.

44. A method according to any one of claims 42 to 43 comprising activating and expanding the double positive CD8+ CD4+ T cells to produce a population of T cells that have a CD8+ single positive phenotype or a CD4+ single positive phenotype.

45. A method according to any one of claims 42 to 44 wherein the T cells specifically bind to cells expressing a target antigen.

46. A method according to claim 45 wherein the target antigen is a tumour antigen.

47. A method according to claim 46 wherein the T cells specifically bind to cancer cells expressing the tumour antigen.

48. A method according to any one of claims 16 to 47 wherein the iPSCs are derived from T cells obtained from a donor individual that are specific for the target antigen.

49. A method according to claim 48 wherein the T cells obtained from the donor individual are tumour-infiltrating lymphocytes (TILs).

50. A method according to any one of claims 1 to 44 wherein the method further comprises introducing heterologous nucleic acid encoding an antigen receptor into the iPSCs, HECs, HPCs, or progenitor T cells.

51. A method according to claim 50 wherein the heterologous nucleic acid encoding the antigen receptor is comprised in an expression vector.

52. A method according to claim 51 wherein the expression vector is a lentiviral vector or adeno-associated viral (AAV) vector.

53. A method according to claim 50 or 51 wherein the heterologous nucleic acid is incorporated into the genome of the iPSCs, HECs, HPCs, or progenitor T cells using a gene editing system.

54. A method according to claim 53 wherein the gene editing system is CRISPR/Cas9 or AAV.

55. A method according to any one of claims 50 to 54 wherein the antigen receptor is a TCR.

56. A method according to claim 55 wherein the TCR is an affinity enhanced TCR.

57. A method according to claim 54 or 55 wherein the TCR binds specifically to an MHC displaying a peptide fragment of a target antigen expressed by cells or specifically binds to a target antigen or peptide thereof expressed by cells independently of MHC presentation.

58. A method according to claim 57 wherein the TCR binds specifically to an MHC displaying a peptide fragment of a tumour antigen expressed by cancer cells or binds specifically to a tumour antigen or peptide fragment thereof expressed by cancer cells independently of MHC presentation.

59. A method according to any one of claims 50 to 54 wherein the antigen receptor is a chimeric antigen receptor (CAR).

60. A method according to claim 59 wherein the CAR binds specifically to a target antigen expressed by cells.

61. A method according to claim 60 wherein the CAR binds specifically to an MHC displaying a peptide fragment of a tumour antigen expressed by cancer cells.

62. A method according to any one of claims 50 to 54 wherein the antigen receptor is a NK cell receptor (NKCR).

63. A method according to claim 62 wherein the NKCR binds specifically to a target antigen expressed by cells.

64. A method according to claim 63 wherein the NKCR binds specifically to an MHC displaying a peptide fragment of a tumour antigen expressed by cancer cells.

65. A haematopoietic induction medium comprising a chemically defined nutrient medium supplemented with one or more differentiation factors, wherein the one or more differentiation factors consist of a differentiation factor that stimulates cKit receptor (CD117) or cKit receptor (CD117) mediated signalling pathways, and a differentiation factor that stimulates VEGFR or VEGFR mediated signalling pathways.

66. A haematopoietic induction medium comprising a chemically defined nutrient medium supplemented with one or more differentiation factors, wherein the one or more differentiation factors consist of (a) a differentiation factor that stimulates cKIT receptor (CD117) mediated signalling pathways, (b) a differentiation factor that stimulates VEGFR mediated signalling pathways, (c) a differentiation factor that stimulates MPL (CD110) mediated signalling pathways (d) a differentiation factor that stimulates FLT3 mediated signalling pathways (e) a differentiation factor that stimulates IGF1R mediated signalling pathways and (f) one or more differentiation factors that display interleukin (IL) activity.

67. A haematopoietic induction medium according to claim 66 wherein the differentiation factor that stimulates MPL mediated signalling pathways is TPO.

68. A haematopoietic induction medium according to claim 66 or claim 67 wherein the differentiation factor that stimulates FLT3 mediated signalling pathways is FLT3L.

69. A haematopoietic induction medium according to any one of claims 66 to 68 wherein the differentiation factor that stimulates IGF1R mediated signalling pathways is IGF1.

70. A haematopoietic induction medium according to any one of claims 66 to 69 wherein the one or more differentiation factors that display interleukin activity are IL-3, IL-6 and optionally IL-7.

71. A haematopoietic induction medium according to any one of claims 65 to 70 wherein the differentiation factor that stimulates cKit receptor (CD117) or cKit receptor (CD117) mediated signalling pathway is SCF.

72. A haematopoietic induction medium according to any one of claims 65 to 710 wherein the differentiation factor that stimulates VEGFR or VEGFR mediated signalling pathways is VEGF.

73. A population of HPCs produced by a method according to any one of claims 1 to 38, wherein the HPCs are thymocytic HPCs having the phenotype CD34+ CD7+.

74. Use of a haematopoietic induction medium according to any one of claims 65 to 7261 for the differentiation of mesoderm cells or HECs into HPCs.

75. A kit for use in the production of HPCs comprising a haematopoietic induction medium according to any one of claims 65 to 72.

76. A kit according to claim 75 further comprising;
a first mesoderm induction medium comprising activin,
a second mesoderm induction medium comprising activin, BMP and FGF, and/or
a third mesoderm induction medium comprising activin, BMP, FGF, and a GSK3 inhibitor,.
